(19) United States
(12) Patent Application Publication
(10) Pub. No.: US 2011/0111976 A1
(43) Pub. Date: May 12, 2011

(54) MICRORNA BIOMARKERS OF TISSUE INJURY

(75) Inventors: Thomas L. Fare, Furlong, PA (US); Philip W. Garrett-Engele, Needham, MA (US); Warren E. Glaab, Doylestown, PA (US); Jason M. Johnson, Needham, MA (US); Omar F. Laterza, New York, NY (US); Lee Lim, San Francisco, CA (US); Steve W. Ludmerer, North Wales, PA (US); David B. Olsen, Lansdale, PA (US); Joseph F. Sina, Maple Glen, PA (US); Frank D. Sistare, Collegeville, PA (US)

(73) Assignee: Merck Sharp & Dohme Corp., Rahway, NJ (US)

(21) Appl. No.: 12/989,246

(22) PCT Filed: Apr. 24, 2009
(86) PCT No.: PCT/US09/41666

§ 371 Date: Jan. 14, 2011

Related U.S. Application Data
(60) Provisional application No. 61/125,448, filed on Apr. 25, 2008.
(60) Provisional application No. 61/210,601, filed on Mar. 19, 2009.

(51) Int. Cl. C40B 30/04; C12Q 1/68; G01N 33/53

(52) U.S. Cl. 506/9; 435/6; 436/94

(57) ABSTRACT

One aspect of the invention generally relates to use of tissue enriched miRNAs as biomarker to estimate tissue damage in a fluid sample. In a second aspect, methods are provided for monitoring a subject who is exposed or might have been exposed to an agent that has a risk of causing tissue injury. In a third aspect, methods are provided for identifying an agent as having a risk of causing tissue injury to a vertebrate subject. In a fourth aspect, kits are provided for practicing the methods of above-listed aspects. The contents of this ABSTRACT are not intended to in anyway limit the scope of the inventions claimed herein.

This application claims priority to U.S. Provisional Patent Application Ser. No. 61/125,448 filed on Apr. 25, 2008, and U.S. Provisional Patent Application Ser. No. 61/210,601 filed on Mar. 19, 2008, each of which is incorporated by reference herein in its entirety.

### FIELD OF THE INVENTION

Aspects and embodiments of the present invention relate generally to methods of detecting tissue injury based on the levels of miRNAs present in fluid samples, such as for example, blood plasma, urine and cerebral spinal fluid.

### BACKGROUND OF THE INVENTION

The following is a short discussion of relevant art pertaining to miRNA biomarkers in vertebrate fluid samples, such as for example whole blood, blood plasma and urine. The discussion is provided only for understanding of the various embodiments of invention that follow. The summary and references cited throughout the specification herein are not an admission that any of the content below is prior art to the claimed invention.

MicroRNAs are endogenous noncoding RNAs of about 22 nucleotides that utilize much of the same cellular machinery harnessed by RNAi. With the discovery of mammalian microRNAs came the discovery that some of them were highly tissue-specific and highly abundant (Lagos-Quintana, M., et al., 2002, Current Biology 12:735). In fact, the effects of some tissue-specific miRNAs are so potent as to be discernable in mRNA profiles (Lim, et al., 2005, Nature 433:769).

The distribution and abundance levels of particular miRNAs have been predicted as being useful to perform diagnosis and/or prognosis of cancer in blood samples from a subject with cancer, in particular breast cancer (US 2007/0054287). However no data was shown in support of this hypothesis. Similarly, Kemppainen et al., have published two posters at the Asuragen, Inc. (Austin, Tex.) website (http://www.asuragen.com/services/library/posters.html, last visited on Apr. 24, 2008) which presented data showing that various miRNAs could be detected in blood and other body fluids, and suggested that miRNAs detected in such fluids might be used as disease biomarkers, however no data is provided in support of this hypothesis.

Marsit et al. (2006 Cancer Res. 66:10843-48), show that the level of miR-222 and miR-22 in blood samples show statistically significant changes between human subjects that have high dietary folate verses those with low dietary folate.

Detection of transaminase enzymes alanine aminotransferase (ALT) and aspartate aminotransferase (AST) in blood plasma is the standard assay for liver toxicity, both in animal models and in clinical studies. Unfortunately, limitations in the sensitivity or specificity of these protein biomarkers can compromise studies. For instance, ALT and AST enzymes are produced by organs besides liver, such as for example, muscle, so changes in these other organs can lead to a false report of tissue injury to liver. Thus, there is a need for new biomarkers for tissue injury to liver and muscle that can either complement or replace existing assays. There is also a similar need for tissue injury biomarkers for other vertebrate tissues and organs, such as, for example, brain, pancreas, kidney, and other organs that are susceptible to tissue injury and for which there is a need for rapid, noninvasive tests of such injury.

### SUMMARY

In one aspect, the present invention provides various embodiments of methods for classifying a subject as having tissue injury. In one embodiment, the method comprises: obtaining a fluid sample from a subject wherein the fluid sample is not obtained directly from the organ or tissue that is suspected of being injured; detecting the presence of a miRNA selected from Table 1 in the fluid sample wherein the presence of the miRNA is detected when the miRNA has a measured value above a threshold value for the selected miRNA; classifying the subject as having tissue injury if the detected miRNA has a measured value above the threshold value; and displaying; or outputting to a user interface device, a computer readable medium, or a local or remote computer system, the classification result.

In a second aspect, the present invention provides various embodiments of methods for monitoring a subject who is exposed, or is thought to have been exposed, to an agent that has a risk of causing tissue injury. In one embodiment, the method comprises: obtaining a fluid sample from the subject; measuring a level of one or more miRNAs in the fluid sample from the subject, wherein the one or more miRNAs are represented by a miRNA selected from Table 1; identifying the subject as being at risk of tissue injury or not based on the measured level of the one or more miRNAs; and displaying; or outputting to a user interface device, a computer readable medium, or a local or remote computer system, the identification result.

In a third aspect, the present invention provides various embodiments of methods for identifying an agent as having a risk of causing tissue injury to a vertebrate subject. In one embodiment, the method comprises: obtaining a fluid sample from a subject exposed to the agent; measuring a level of one or more miRNAs in the fluid sample from the subject, wherein the one or more miRNAs are represented by a miRNA selected from Table 1; identifying the agent as having a risk of causing tissue injury based on the measured level of the one or more miRNAs.

In a third aspect, the present invention provides various kits for use in the practice of any of the inventive methods disclosed herein, including, for example, methods for classifying a subject as having tissue injury; methods for monitoring a subject is exposed to an agent that has a risk of causing tissue damage; and methods for identifying an agent as having a risk of causing tissue injury to a vertebrate subject.

### BRIEF DESCRIPTION OF THE DRAWINGS

FIG. 1 shows miRNAs that have tissue-enriched expression levels.

FIG. 2 shows miRNA and standard protein plasma biomarker levels in plasma from rats dosed with muscle and liver toxicants.

FIG. 3 shows a receiver operating characteristic (ROC) curve for sensitivity and specificity of using ALT, AST, and miR-122 measurements to predict liver histopathology.

FIG. 4 shows ALT or AST protein plasma biomarker levels plotted verses miR-122 plasma copy number in HCV genotype 1a infected subjects

### DETAILED DESCRIPTION OF THE INVENTION

This section presents a detailed description of the many different aspects and embodiments that are representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure the ordinary skilled artisan can identify and employ other components and methodology useful for practicing the various embodiments of the present invention.

### I. DEFINITIONS

Unless defined otherwise, all technical and scientific terms used herein have the meaning commonly understood by one of ordinary skill in the art to which this invention belongs. Practitioners are particularly directed to Sambrook et al. (1989) Molecular Cloning: A Laboratory Manual, 2d ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. For example, a concentration of “about” 100 mM of Tris can encompass an amount of 100 mM .+−.5 mM, if 5 mM represents the collective error bars in arriving at that concentration. Thus, any measurement or amount referred to in this application can be used with the term “about” if that measurement or amount is susceptible to errors associated with calibration or measuring equipment, such as a scale, pipetteman, pipette, graduated cylinder, etc. The term “about”, when used in reference to a number, is generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “tissue injury” refers to any damage to cells in an organ or tissue that causes leakage of cellular components from cells. For example, tissue injury may be caused by any form of chemical or physical agents, such as, drugs, environmental toxicants, or any other substance that contacts a subject and results directly or indirectly, in damage to the cells of the organ or tissue. Also included, is cellular damage that results from successful therapeutic treatment of a subject, such as for example, the treatment of a tumor which results in induction of apoptosis. Similarly, tissue injury might be the result of a physical agent such as, for example, exposure to an environmental condition such as an hypoxic condition or air or water pollution. Alternatively, the physical agent might be a physical trauma event, whether self-imposed or not, such as for example, exercise, smoking, blunt force trauma, stroke, etc.

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs and miRNAs. For clarity, the term gene generally refers to a portion of a nucleic acid that encodes a protein or functional RNA; however, the term may optionally encompass regulatory sequences. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

As used herein, the term “microRNA species”, “microRNA”, “miRNA”, or “miR” refers to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15-25, or 20 to 30 nucleotides in length, and more often between about 16 to 24, 17 to 23, 18 to 22, 19 to 21 or, 21 to 24 nucleotides in length. MicroRNAs include processed sequences as well as corresponding long primary transcripts (pri-miRNAs) and processed precursors (pre-miRNAs). Some microRNA molecules function in living cells to regulate gene expression via RNA interference. A representative set of microRNA species is described in the publicly available miRBase sequence database as described in Griffith-Jones et al., Nucleic Acids Research 32:D109-D111 (2004) and Griffith-Jones et al., Nucleic Acids Research 34:D140-D144 (2006), accessible on the World Wide Web at the Wellcome Trust Sanger Institute website.

As used herein, the term “isolated” in the context of an isolated nucleic acid molecule, is one which is altered or removed from the natural state through human intervention. For example, an RNA naturally present in a living animal is not “isolated.” A synthetic RNA or dsRNA or microRNA molecule partially or completely separated from the coexisting materials of its natural state, is “isolated.” Thus, an miRNA molecule which is deliberately delivered to or expressed in a cell is considered an “isolated” nucleic acid molecule.

As used herein, “RNA” refers to a molecule comprising at least one ribonucleotide residue. The term “ribonucleotide” means a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of an RNAi agent or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “essentially complementary” with reference to microRNA target sequences refers to microRNA target nucleic acid sequences that are longer than 8 nucleotides that are complementary (an exact match) to at least 8 consecutive nucleotides of the 5′ portion of a microRNA molecule from nucleotide positions 1 to 10, (also referred to as the “seed region”), and are at least 65% complementary (such as at least 70%, at least 75%, at least 80%, at least 85%, at least 90%, at least 95%, or at least 96% identical) across the remainder of the microRNA target nucleic acid sequence as compared to a naturally occurring mir-34 family member. The comparison of sequences and determination of percent identity and similarity between two sequences can be accomplished using a mathematical algorithm of Karlin and Altschul (1990, PNAS 87:2264-2268), modified as in Karlin and Altschul (1993, PNAS 90:5873-5877). Such an algorithm is incorporated into the NBLAST and XBLAST programs of Altshcul et al, (1990 J. Mol. Biol. 215:403-410).

As used herein, the terms “measuring miRNA levels,” “obtaining an miRNA expression level” and the like, includes methods that quantify a miRNA expression level. Thus, an assay which provides a “yes” or “no” result without necessarily providing quantification, of an amount of expression is an assay that “measures expression” as that term is used herein. Alternatively, a measured or obtained expression level may be expressed as any quantitative value, for example, a fold-change in expression, up or down, relative to a control transcript or miRNA or relative to the same transcript or miRNA in another sample, or a log ratio of expression, or any visual representation thereof, such as, for example, a “heatmap” where a color intensity is representative of the amount of gene expression detected. Exemplary methods for detecting the level of expression of a miRNA include, but are not limited to, Northern blotting, dot or slot blots, nuclease protection, RT-PCR, microarray profiling, and the like.

As used herein, the phrase “fold difference” refers to a numerical representation of the magnitude difference between a measured value and a reference value for one or more of the miRNA biomarkers of the present invention. Fold difference may be calculated mathematically by division of the numeric measured value with the numeric reference value.

As used herein, a “reference value” or “control value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from a subject who has suffered tissue injury, or a value obtained from a sample from a subject other than the subject being tested, or a “normal” subject, that is an individual who has not suffered tissue injury. Alternatively, the reference value can be based on a large number of samples, such as from subjects who are all known, or thought, to have suffered tissue injury or are from normal individuals who are known, or thought, to not have tissue injury or based on a pool of samples including or excluding the sample to be tested.

The term “normalized” with regard to a miRNA or a gene expression product refers to the level of the primary transcript, gene expression product or processed miRNA relative to the mean levels of transcripts/products/miRNAs of a set of reference genes or miRNAs, wherein the reference genes or miRNAs are either selected based on their minimal variation across, subjects, tissues or treatments (“constitutive genes or miRNAs”), or the reference genes or miRNAs are the totality of tested genes and/or miRNAs. In the latter case, which is commonly referred to as “global normalization”, it is important that the total number of tested genes or miRNAs be relatively large, i.e., greater than 10, 20, 30, 40 or 50 transcription products. Specifically, the term “normalized” with respect to an RNA transcript refers to the transcript level relative to the mean of transcript levels of a set of reference genes. More specifically, the mean level of an RNA transcript as measured by TaqMan (D RT-PCR refers to the Ct value minus the mean Ct values of a set of reference RNA transcripts.

The terms “threshold value” or “defined threshold” are used interchangeably and refer to the level of a miRNA or gene product in question above which the miRNA or gene product serves as a biomarker, in a fluid sample, for tissue injury. The threshold typically is defined experimentally from experimental or clinical studies. The expression threshold can be selected based on either the need for maximum sensitivity (for example use of an miRNA that is not specific to detection tissue injury in only a single organ or tissue system of the subject), or for maximum selectivity (for example to detect tissue injury in only one tissue or organ system of the subject), or for minimum error.

As used herein, reference to “at least one,” “at least two,” “at least five,” etc., of the miRNAs listed in any particular table or designated gene set means any one or any and all combinations of the miRNAs listed.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

As used herein, a “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

As used herein, “subject”, refers to a vertebrate organism, including but not limited to, an animal, such as a cow, a pig, a mouse, a rat, a chicken, a cat, a dog, etc., and is usually a mammal, such as a human, monkey, ape, or baboon.

As used herein, “present invention” is not intended to in any way limit the claimed invention, and is used herein to refer to any one of the many different aspects, embodiments of inventions described and/or claimed herein, including or any combination thereof.

### II. ASPECTS AND EMBODIMENTS OF THE INVENTION

The present invention broadly relates to the use of miRNAs as biomarkers for detection of tissue injury or to classify agents regarding the risk of the agent causing tissue injury. Some miRNAs have been found to have very specific patterns of gene expression, that is they are found only in one or a few tissues or organs. An overview of the abundances of miRNAs in the human body atlas (Example 1) shows that, unlike other plentiful noncoding RNAs such as ribosomal and spliceosomal RNAs, miRNAs have a diversity of expression patterns that befits their role as important RNA regulators. Like mRNAs, they show a gradient of expression patterns ranging from extremely tissue-specific to nearly ubiquitous.

Fluid systems in vertebrates come in contact with many different body tissues and organs. For example, blood circulates throughout the body, the cerebral spinal fluid circulates throughout the brain and spinal cord, and urine is a fluid that results from function of the kidney. Each of these body fluids has the opportunity to accumulate and transport small quantities of cellular components, including miRNAs, that are released due to tissue injury. Should an tissue undergo a physical trauma or be subjected to a chemical agent that induces tissue injury, then miRNAs specific to that tissue or organ might undergo an increase in concentration within the corresponding fluid associated with the injured tissue.

It is contemplated that any embodiment discussed in this specification can be implemented with respect to any method, kit, reagent, or composition of the invention, and vice versa. Furthermore, compositions of the invention can be used to achieve methods of the invention. For example, any of the embodiments of the present invention can be used on a wide variety of vertebrate subjects, including for example, dog, cat, pig, rat, mouse, monkey, baboon, ape, human, bison, cow, horse, and chicken.

In one aspect of the invention, a method is provided for classifying a vertebrate subject as having tissue injury. In one embodiment, the method comprises obtaining a fluid sample from the subject and detecting the presence of a miRNA selected from Table 1 in the fluid sample, wherein the presence of the miRNA is detected when the miRNA has a measured value above a threshold value for the miRNA. The subject is then classified as having suffered tissue injury if the detected miRNA has a measured value above the threshold value.

TABLE 1
miRNA Sequences

| | | Seq |
|---|---|---|
| Human | | ID |
| miRNA | Sequence | No: |
| miR-122a | UGGAGUGUGACAAUGGUGUUUGU | 1 |
| miR-128a | UCACAGUGAACCGGUCUCUUUU | 2 |
| miR-128b | UCACAGUGAACCGGUCUCUUUC | 3 |
| miR-129 | CUUUUUGCGGUCUGGGCUUGC | 4 |
| miR-133a | UUGGUCCCCUUCAACCAGCUGU | 5 |
| miR-133b | UUGGUCCCCUUCAACCAGCUA | 6 |
| miR-137 | UAUUGCUUAAGAAUACGCGUAG | 7 |
| miR-301 | CAGUGCAAUAGUAUUGUCAAAGC | 8 |
| miR-302c | UAAGUGCUUCCAUGUUUCAGUGG | 9 |
| miR-31 | GGCAAGAUGCUGGCAUAGCUG | 10 |
| miR-323 | GCACAUUACACGGUCGACCUCU | 11 |
| miR-330 | GCAAAGCACACGGCCUGCAGAGA | 12 |
| miR-346 | UGUCUGCCCGCAUGCCUGCCUCU | 13 |
| miR-34b | UAGGCAGUGUCAUUAGCUGAUUG | 14 |
| miR-34c | AGGCAGUGUAGUUAGCUGAUUGC | 15 |
| miR-375 | UUUGUUCGUUCGGCUCGCGUGA | 16 |
| miR-9* | UAAAGCUAGAUAACCGAAAGU | 17 |
| miR-124a | UUAAGGCACGCGGUGAAUGCCA | 18 |
| miR-138 | AGCUGGUGUUGUGAAUCAGGCCG | 19 |
| miR-149 | UCUGGCUCCGUGUCUUCACUCC | 20 |
| miR-153 | UUGCAUAGUCACAAAAGUGA | 21 |
| miR-181c | AACAUUCAACCUGUCGGUGAGU | 22 |
| miR-18a | UAAGGUGCAUCUAGUGCAGAUA | 23 |
| miR-205 | UCCUUCAUUCCACCGGAGUCUG | 24 |
| miR-216 | UAAUCUCAGCUGGCAACUGUG | 25 |
| miR-217 | UACUGCAUCAGGAACUGAUUGGAU | 26 |
| miR-338 | UCCAGCAUCAGUGAUUUUGUUGA | 27 |
| miR-383 | AGAUCAGAAGGUGAUUGUGGCU | 28 |

In some embodiments, the classification result is displayed as a written report that, optionally, provides a summary of the detected miRNA levels and/or an identification of the tissue that has been most likely injured. In still other embodiments, the classification result is outputted to a user interface device, a computer readable medium, or a local or remote computer system.

In some embodiments, the threshold value is obtained by obtaining a second measure value of the miRNA in a control fluid sample obtained from a control vertebrate subject that has not suffered tissue injury. Alternatively, the threshold value is obtained by obtaining a second measure value of the miRNA in a control fluid sample obtained from a control vertebrate subject that is known to have suffered tissue injury. In some embodiments, where the control fluid sample is obtained from a subject known to have suffered tissue injury, detection of a measured level that is below the threshold level indicates that little or no tissue injury has occurred, while detection of a measured level that is at or above the threshold level indicates that tissue injury has occurred. In some instances, control subjects are further evaluated to determine the degree of tissue injury using art recognized protein biomarkers or histopathology analysis using samples of the fluid and/or injured tissue.

In yet other embodiments, the fluid sample is not obtained directly from the injured tissue but is rather obtained from whole blood, blood plasma, blood serum, cerebrospinal fluid, saliva, seminal fluid, breast nipple aspirate, or urine, or combinations thereof.

In one specific embodiment, the miRNA is miR-122A and the fluid sample is obtained from whole blood, blood plasma, or blood serum. In this embodiment, if miR-122A is detected in the fluid sample above a threshold level, then the subject is classified as having suffered damage to the liver.

In another embodiment, the miRNA is miR-10B, miR-10A, miR-196A, or miR-196B, and the fluid sample is obtained from urine. If one or more of the above listed miRNAs is detected in the fluid sample above a threshold level, then the subject is classified as having suffered damage to the kidney.

In another embodiment, the miRNA is miR-216, miR-217 or miR375, and the fluid sample is obtained from whole blood, blood plasma, or blood serum. If one or more of the above listed miRNAs is detected in the fluid sample above a threshold level, then the subject is classified as having suffered damage to the pancreas.

In yet another embodiment, the miRNA is miR-133, miR-1 or miR-206, and the fluid sample is obtained from whole blood, blood plasma, or blood serum. If one or more of the above listed miRNAs is detected in the fluid sample above a threshold level, then the subject is classified as having suffered damage to the skeletal muscle or cardiac muscle.

In another embodiment, the miRNA is miR-124a, miR-9*; miR-9, miR-219, miR-137, miR-323, miR-330, miR-346, miR-153, miR-128a, miR-338, miR-7, miR-329, miR-132, miR-433, miR-128B, miR-138, miR-212, miR-340, miR-149, miR-181, miR-383, or miR-129, and the fluid sample is obtained from whole blood, blood plasma, blood serum, or cerebrospinal fluid. If one or more of the above listed miRNAs is detected in the fluid sample above a threshold level, then the subject is classified as having suffered damage to the brain.

In some instances, the inventive methods are used to evaluate whether the subject has been exposed to an agent that is known to cause tissue injury in vertebrates. In one embodiment, a control fluid sample is obtained from the subject prior to the subject being exposed to the agent. After exposure to the agent a fluid sample is obtained and the measured level of miRNA biomarker is compared to the level in the control fluid sample. For example, the subject may be undergoing treatment with an agent that is known to cause, in some individuals, damage to the liver. In this example, the subject would be monitored before and after treatment with the agent by obtaining a blood sample and then detecting the presence or absence of miR-122 in the sample. As shown in Examples 2 and 3, the level of miR-122 in blood is typically very low, however, miR-122 levels increase a short time after exposure to agents that are known to induce liver damage.

In some embodiments, the same methods are used to evaluate the effects of an infectious agent, such as a virus or bacterium, on a subject wherein the infectious disease is known or suspected of causing tissue injury. For example, certain forms of hepatitis are caused by viral or bacterial infections. The degree of liver damage induced by the infection may be monitored by measurement of miR-122 level in the fluid sample. In some instances, the response of a subject to treatment can be monitored by regular evaluation of miRNA levels in the fluid sample.

In a second aspect of the invention, a method is provided for monitoring a subject who is exposed or might have been exposed to an agent that has a risk of causing tissue injury. In one embodiment, the method comprises obtaining a fluid sample from the subject exposed to the agent, measuring a level of one or more miRNAs in the fluid sample, wherein the one or more miRNA is represented by a miRNA listed in Table 1. The subject is then identified as being at risk of tissue injury, or not being at risk of tissue injury, based on the measured level of the selected one or more miRNAs. In some embodiment, the measured level of miRNA is compared to a threshold value, if the measure miRNA value is above the threshold value, then the subject is identified as being at risk of tissue injury. Alternatively, if the measure value is below the threshold value, then the subject is identified as not being at risk of tissue injury.

In some embodiments, the identification result is displayed or outputted as a written report that, optionally, provides a listing of the measured miRNA levels. In still other embodiments, the identification result is outputted to a user interface device, a computer readable medium, or a local or remote computer system.

In some embodiments, the threshold value is set by obtaining a second measure value of the miRNA in a control fluid sample obtained from a control vertebrate subject that has not been exposed to the agent. In some instances, the control subject is the same individual as the subject, however, the control fluid sample is obtained prior to exposure to the agent. Alternatively, the threshold value is set by obtaining a second measured value of the miRNA in a control fluid sample obtained from a control vertebrate subject that has been exposed to the agent in a amount sufficient to cause tissue injury. In some instances, control subjects are further evaluated to determine the degree of tissue injury using art recognized protein biomarkers or histopathology using a fluid sample or a sample obtained directly from the injured tissue.

In yet other embodiments, the said fluid sample is not obtained directly from the injured tissue or the tissue that is suspected of being injured, but is obtained from whole blood, blood plasma, blood serum, cerebrospinal fluid, saliva, seminal fluid, breast nipple aspirate, or urine, or combinations thereof.

In one specific embodiment, where the agent is known to have a risk of causing tissue injury to the liver, the selected miRNA is miR-122A and the fluid sample is obtained from whole blood, blood plasma, or blood serum. In this embodiment, if miR-122A is detected in the fluid sample above a threshold level, then the subject is identified as being at risk of suffered tissue injury to the liver and exposure to the agent should be altered, i.e., stopped or the exposure lowered. Exemplary agents include, any compound or agents known to have a risk of inducing tissue injury to the liver. Such agents include, alcohol, acetaminophen, nefazodone HCL, darnuavir, interferon beta-1a, telithromycin, bromobenzene, carbon tetrachloride, and tricholorobromomethane.

In another embodiment, where the agent is known to have a risk of causing damage to the kidney, then the selected miRNA is miR-10B, miR-10A, miR-196A, or miR-196B, and the fluid sample is obtained from urine. In this embodiment, if one or more of the above listed miRNAs is detected in the fluid sample above a threshold level, then the subject is identified as being at risk of suffered tissue injury to the kidney and exposure to the agent should be altered, i.e., stopped or the exposure lowered. Exemplary agents include any compound or agents known to have a risk of inducing tissue injury to the kidney. Such agents include, cisplatin, cyclosporin A, carbapenem A, gentamicin, adriamycin, rosiglitazone, and pioglitazone.

In another embodiment, where the agent is known to have a risk of causing tissue injury to the pancreas, then the selected miRNA is miR-216, miR-217 or miR375, and the fluid sample is obtained from whole blood, blood plasma, or blood serum. In this embodiment, if one or more of the above listed miRNAs is detected in the fluid sample above a threshold level, then the subject is identified as being at risk of suffered tissue injury to the pancreas and exposure to the agent should be altered, i.e., stopped or the exposure lowered. Exemplary agents include any compound or agents known to have a risk of inducing tissue injury to the pancreas. Such agents include, rosiglitazone, pioglitazone, and Staphylococcal α-toxin.

In another embodiment, where the agent is known to have a risk of causing tissue injury to muscle, such as skeletal muscle or cardiac muscle, then selected miRNA is miR-133, miR-1 or miR-206, and the fluid sample is obtained from whole blood, blood plasma, or blood serum. In this embodiment, if one or more of the above listed miRNAs is detected in the fluid sample above a threshold level, then the subject is identified as being at risk of suffered tissue injury to muscle and exposure to the agent should be altered, i.e., stopped or the exposure lowered. Exemplary agents include any compound or agents known to have a risk of causing tissue injury to muscle. Such agents include, statins, chloroquine, ephedrine, 2,3,5,6-tetramethyl-p phenylenediamine, doxorubicin, allylamine, and isoproterenol,

In another embodiment, where the agent is known to have a risk of causing tissue injury to the brain, then the selected miRNA is miR-124a, miR-9*; miR-9, miR-219, miR-137, miR-323, miR-330, miR-346, miR-153, miR-128a, miR-338, miR-7, miR-329, miR-132, miR-433, miR-128B, miR-138, miR-212, miR-340, miR-149, miR-181, miR-383, or miR-129, and the fluid sample is obtained from whole blood, blood plasma, blood serum, or cerebrospinal fluid. In this embodiment, if one or more of the above listed miRNAs is detected in the fluid sample above a threshold level, then the subject is identified as being at risk of tissue injury to the brain and exposure to the agent should be altered, i.e., stopped or the exposure lowered. Exemplary agents include any compound or agents known to have a risk of inducing brain damage. Such agents include, lead, mercury, manganese, 3,4-methylenedioxymethylamphetamine, and tipranavir when co-administered with ritonavir.

In a third aspect of the invention, a method is provided for identifying an agent as having a risk of causing tissue injury to a vertebrate subject. In one embodiment, the method comprises obtaining a fluid sample from the subject exposed to the agent, measuring a level of one or more miRNAs in the fluid sample from the subject, wherein the one or more miRNA is represented by a miRNA listed in Table 1. The agent is then identified as having a risk of causing tissue injury based on the measured level of the one or more miRNAs in the fluid sample. In one embodiment, the measured level of miRNA is compared to a threshold value, if the measure miRNA value is above the threshold value then the agent is identified as being at risk of causing tissue injury.

In some embodiments, the identification result is displayed or outputted as a written report that, optionally, provides a listing of the measured miRNA levels. In still other embodiments, the identification result is outputted to a user interface device, a computer readable medium, or a local or remote computer system.

In other embodiments, the threshold value is set by obtaining a second measured value of the miRNA in a control fluid sample obtained from a control vertebrate subject that has been exposed to an agent in a amount that is known cause tissue injury. Alternatively, the threshold value is set by obtaining a second measured value of the miRNA in a control fluid sample obtained from a control vertebrate subject that has not been exposed to an agent that is known to cause tissue injury. In some instances, control subjects are further evaluated to determine the degree of tissue injury using art recognized protein biomarkers or histopathology.

In yet other embodiments, the fluid sample is not obtained directly from the injured tissue or the tissue that is suspected of being injured but is rather obtained from whole blood, blood plasma, blood serum, cerebrospinal fluid, saliva, seminal fluid, breast nipple aspirate, or urine, or combinations thereof.

In one specific embodiment, where the agent is being tested to determine if it has a risk of causing tissue injury to the liver, the selected miRNA is miR-122A and the fluid sample is obtained from whole blood, blood plasma, or blood serum. In this embodiment, if miR-122A is detected in the fluid sample above a threshold level, then the agent is identified as having a risk of causing tissue injury to the liver.

In another embodiment, where the agent is being tested to determine if it has a risk of causing tissue injury to the kidney, the selected miRNA is miR-10B, miR-10A, miR-196A, or miR-196B, and the fluid sample is obtained from urine. In this embodiment, if one or more of the above listed miRNAs is detected in the fluid sample above a threshold level, then the agent is identified as having a risk of tissue injury to the kidney.

In another embodiment, where the agent is being tested to determine if it has a risk of causing tissue injury to the pancreas, the selected miRNA is miR-216, miR-217 or miR375, and the fluid sample is obtained from whole blood, blood plasma, or blood serum. In this embodiment, if one or more of the above listed miRNAs is detected in the fluid sample above a threshold level then the agent is identified as having a risk of causing tissue injury to the pancreas.

In another embodiment, where the agent is being tested to determine if it has a risk of causing tissue injury to muscle, such as skeletal muscle or cardiac muscle, the selected miRNA is miR-133, miR-1 or miR-206, and the fluid sample is obtained from whole blood, blood plasma, or blood serum. In this embodiment, if one or more of the above listed miRNAs is detected in the fluid sample above a threshold level, then the agent is identified as having a risk of causing tissue injury to muscle.

In another embodiment, where the agent is being tested to determine if it has a risk of causing tissue injury to the brain, the selected miRNA is miR-124a, miR-9*; miR-9, miR-219, miR-137, miR-323, miR-330, miR-346, miR-153, miR-128a, miR-338, miR-7, miR-329, miR-132, miR-433, miR-128B, miR-138, miR-212, miR-340, miR-149, miR-181, miR-383, or miR-129, and the fluid sample is obtained from whole blood, blood plasma, blood serum, or cerebrospinal fluid. In this embodiment, if one or more of the above listed miRNAs is detected in the fluid sample above a threshold level, then the agent is identified as having a risk of causing tissue injury to the brain.

In some embodiments, the method is repeated using a plurality of different agents and a plurality of different subjects and the plurality of agents are rank order listed based upon the risk of each agent causing tissue injury.

This aspect of the invention may be used to evaluate the risk of tissue injury for a wide variety of therapeutic agents, including, but not limited to, proteins, such as antibodies, enzymes, etc., nucleic acids, such as antisense oligonucleotides, siRNAs, miRNAs, miRNA antimers, etc., and small molecule compounds.

Alternatively, the inventive methods can be used to access the risk of exposure of a subject to toxic or infectious agents including, for example, viruses, bacteria, household cleaners, paints, heavy metals and other chemicals, food additives. Alternatively, the inventive methods might be used to monitor water quality, food quality or any other application where it is desirable to determine the risk of exposure to an agent.

It is a further aspect of the present invention to provide a kit for use in the practice of any of the inventive methods disclosed herein, including, for example, determining or predicting whether a subject has suffered tissue injury, such as for example, as a side effect of therapeutic treatment with an agent or as the result of physical trauma, for example stroke.

Embodiments of this aspect contemplate a kit comprising a pair of primers for nucleic acid amplification and/or a probe for hybridization to a miRNA biomarker of the present invention that is predictive of tissue injury to one or more tissue or organ, in a fluid sample obtained from a subject; and instructional material for use of the primers and/or the probe to determine the presence or the absence of the miRNA in the fluid sample. Alternatively, provided in the kit are one or more microarrays, e.g., oligonucleotide microarrays or cDNA microarrays comprising probes that hybridize to the predictive miRNA biomarkers and instruction for use of the microarray. In some embodiments the kits, comprising probes for miRNA biomarkers of tissue or organ damage, such as for example, miR-122A, miR-10B, miR-10A, miR-196A, miR-196B, miR-133, miR-1, miR-206, miR-124a, miR-9*; miR-9, miR-219, miR-137, miR-323, miR-330, miR-346, miR-153, miR-128a, miR-338, miR-7, miR-329, miR-132, miR-433, miR-1288, miR-138, miR-212, miR-340, miR-149, miR-181, miR-383, miR-129, miR-216, miR-217 or miR375, or a combination thereof; and, in suitable containers, control or reference samples to compare the patient measured miRNA values to; and instructions for use.

In a different aspect, the invention concerns a method of preparing a personalized miRNA tissue injury profile for a subject or an agent, comprising the steps of: (a) subjecting RNA extracted from a fluid sample obtained from the subject exposed to an agent to miRNA expression analysis; (b) determining the expression level of one or more of the miRNA tissue injury biomarkers disclosed herein, wherein the expression level is normalized against one or more control miRNAs, or one or more control genes, and optionally is compared to the level of miRNA or control genes found in a tissue injury reference set that provides threshold values for a plurality of tissue injury miRNAs in each fluid type in association with a type of tissue injury; and (c) creating a report summarizing the data obtained by the miRNA expression analysis. The report may, for example, optionally include prediction of the degree of tissue injury present in the subject and/or likely causative agents. Those skilled in the art should understand that in the methods of the invention, the “providing a biological sample from a subject” is not a necessary feature to exploit the invention. Therefore, some embodiments of the invention may exclude this step.

In some embodiments of the present invention, the measured value is obtained using a polymerase chain reaction method, a Northern blot hybridization method, or a nucleotide microarray hybridization method, a single-molecule method or any other method that is capable of providing a measured level (either as a quantitative amount or as an amount relative to a standard or control amount, i.e., a ratio or a fold-change) of a microRNA within a cell or tissue sample.

Fluid samples can undergo any of a variety of sample preparation procedures known in the art to prepare nucleic acid molecules for analysis, such as, for example, the methods referenced in the Examples. In some embodiments, the fluid sample undergoes a heat lysis treatment, and microRNA is quantified thereafter. In other embodiments, for example when the fluid sample is blood, the fluid sample can be collected in a commercially available Tempus Tube® from Applied Biosystems, and microRNA quantified thereafter. In some circumstances, the fluid samples may be kept at room temperature. In other instances, the samples will be kept at less than about 10 degrees centigrade, or less than about zero degrees centigrade, or less than about minus 20 degrees centigrade or less than about minus 60 degrees centigrade. For example, the samples may be maintained on water ice, dry ice or in liquid nitrogen, until nucleic acid is extracted from the sample. In some embodiments, various other sample preparation procedures commonly employed in the art of molecular biology can be employed, including for example the mirVana micro RNA isolation kit (commercially available from Ambion) and the 6100 nucleic acid sample prep products commercially available from Applied Biosystems.

In some embodiments, the comparison of the measured value and the reference or control value includes calculating a fold difference between the measured value and the reference value. In some embodiments the measured value is obtained by measuring the level of miRNA biomarker in the sample, while in other embodiments the measured value is obtained from a third party. In certain embodiment, measured expression level of miRNA evaluated to determine that it is statistically significant, e.g., has a p-value of <0.05 or <0.1.

As noted, supra, it is understood that a miRNA level can be obtained by any method and that the measurement level can be a absolute level, i.e., intensity level from a microarray, a ratio, i.e., compared to a control level either of a reference transcript or the miRNA itself, or a log ratio. For example, the pre-determined level may comprises performing the same miRNA measurement determination in a control sample of cells and comparing the same to a fluid sample obtained from subject who is being tested for tissue injury. In another non-limiting example, the control sample may be a plurality of samples obtained from a single or a plurality of subjects that have not been exposed to an agent under evaluation for tissue injury or a sample of cells from a subject exposed to a known tissue injury agent. Other control samples are within the level of skill level of a skilled laboratory technician, clinician or scientist.

The absolute or relative amounts of expression of one or more genes, including miRNA genes, or the pattern of expression of multiple genes, can be measured by any method for measuring gene expression. For example, reverse transcription followed by PCR (referred to as RT-PCR) can be used to measure gene expression. RT-PCR involves the PCR amplification of a reverse transcription product, and can be used, for example, to amplify very small amounts of any kind of RNA (e.g., mRNA, rRNA, tRNA). RT-PCR is described, for example, in Chapters 6 and 8 of The Polymerase Chain Reaction, Mullis, K. B., et al., Eds., Birkhauser, 1994, the cited chapters of which publication are incorporated herein by reference.

A sensitive quantitative PCR (qPCR) methods has been developed to improve the detection and measurement of miRNA transcripts (Raymond et al, 2005, RNA 11:1737; WO 2006/081282). The above-cited publications by Raymond et al., describe the methods and reagents needed to quantitatively determine miRNA levels in a variety of vertebrate species. Instructions and reagents, including qPCR primers for a large number of miRNAs in human, rat and mouse are available from Exiqon, Inc. (Woburn, Mass.).

Again by way of example, kits, reagents and instructions are available from Exiqon, Inc. (Woburn, Mass.) that utilize LNA probes for detection of miRNAs from a large number of vertebrate species using Northern blot hybridization methods.

By way of further example, DNA microarrays can be used to measure gene expression, in particular miRNA expression levels. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, 1996, BioEssays 18:427). Exemplary microarrays and methods for their use in the detection of miRNA levels in human, rat and mouse are available from Agilent Technologies (Santa Clara, Calif.) and Exiqon, Inc.

Alternatively, a variety of other method can be used to determine the level of a miRNA in a sample. Neely et al. (2006, Nat. Methods 3:41-46) provide a single-molecule method for the quantitation of microRNA gene expression. Geiss et al. (2008 Nat. Biotechnol. 26:317-325) described a multiplex NanoString nCounter gene expression system that has reported potential to perform multiplex miRNA profiling (Forting and Surrey Nat. Biotechnol. 26:293-94). Bloch (US 2007/0054287) describes a stem-loop primer PCR method to measure miRNA levels in order to diagnose cancer and other biological conditions.

Art-recognized statistical techniques can be used to compare the levels of expression of individual genes, or groups of genes, to identify genes which exhibit statistically significantly different expression levels in RNA samples prepared from a fluid sample obtained from a subject. Thus, for example, a t-test can be used to determine whether the mean value of repeated measurements of the level of expression of a particular gene or miRNA is significantly different in a sample from a subject as compared to control sample. Similarly, Analysis of Variance (ANOVA) can be used to compare the mean values of two or more populations of genes or miRNAs in sample from a subject compared to a control sample to determine whether the means are significantly different.

In some embodiments, microRNA expression levels can be normalized to the number of cells directly measured in the test sample by conventional means. In other embodiments, it may be easier and cheaper to normalize to the expression levels measured for housekeeping small RNA, such as described in US 2007/0054287, in parallel to those for the microRNA tissue injury biomarker, which in turn can be calibrated on a per-cell basis in separate reactions. In some embodiments, the endogenous control sequence can be a microRNA that is normally abundantly expressed in fluid sample but is not expressed in the tissue or organ being evaluated for tissue injury.

In some embodiments, the quantity of the endogenous control miRNA correlates negatively with the presence of tissue injury to the target organ or tissue. For example, the sensitivity and/or accuracy of the method may be improved be evaluating the quantity of the target miRNA, that is the miRNA that is a biomarker for tissue injury to the target organ or tissue, and quantity of the control miRNA, that is a miRNA known to be present in organs or tissues that are not being evaluated for tissue injury, and comparing the measure quantities to one another.

### EXAMPLES

Examples are provided below to further illustrate different features and advantages of the present invention. The examples also illustrate useful methodology for practicing the invention. These examples do not limit the claimed invention.

### Example 1

### Tissue Specific miRNAs

This examples shows the identification of tissue-specific miRNAs that are candidate miRNAs to detect tissue injury in fluid samples. A quantitative PCR method (Raymond et al., 2005, RNA 11:1737; WO 2006/081282) was used to detect and quantitate ˜200 different miRNAs on a robotic platform. MicroRNA levels were measured from 23 human tissues and cell lines (the miRNA body atlas). Abundance measurements for miRNAs in the body atlas. Measurements are estimates of the number of miRNA copies per a cell, assuming 20 pg of total RNA per a cell and using DNA standards.

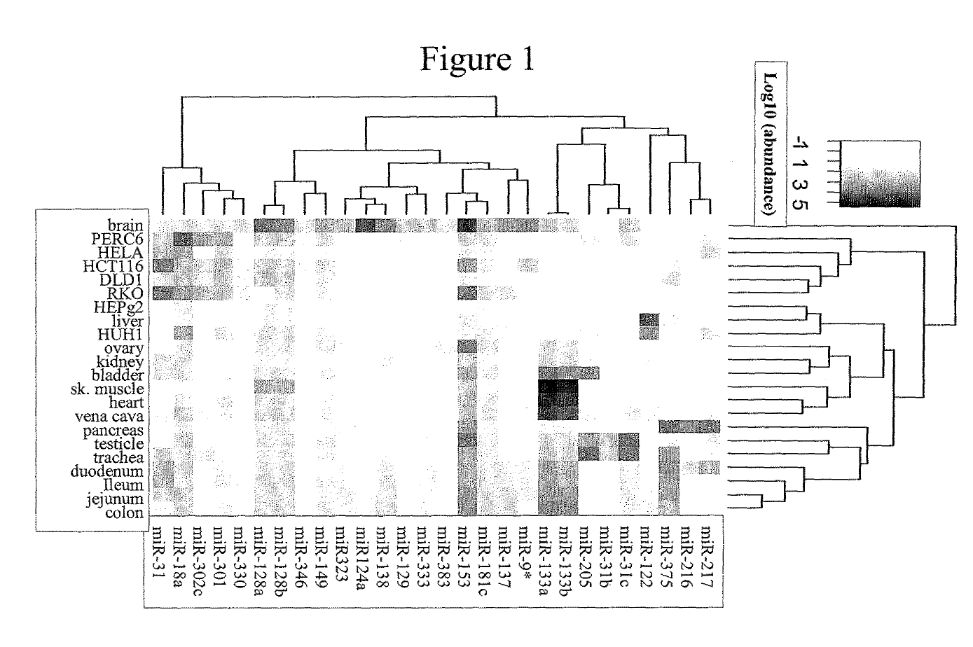

An overview of the abundances of miRNAs in across these tissues (data not shown) shows that, unlike other plentiful noncoding RNAs such as ribosomal and spliceosomal RNAs, miRNAs have a diversity of expression patterns that befits their role as important RNA regulators. Like mRNAs, they show a gradient of expression patterns ranging from extremely tissue-specific to nearly ubiquitous. FIG. 1 and Tables 2A and 2B shows a view of the most tissue-specific miRNAs, as calculated using a simple standard deviation metric. The tissue specificity of a miRNA was measured by dividing the standard deviation across samples by the mean across samples. Shown here are 28 miRNA species for which this ratio was greater than 2, and which were expressed in a tissue at a level of at least 50 copies per cell. For the ratio calculation, intestinal samples were averaged (colon, duodenum, ileum, jejunum), as were muscle (skeletal, heart, and bladder) and cell lines. ΔCt values were converted to copy number by comparison with standard curves generated by use of defined inputs of single stranded mature miRNAs.

TABLE 2A
Copies per cell in twelve tissues of 28 miRNAs determined using real time PCR analysis.

| | | | | | | | | | | vena | | |
|---|---|---|---|---|---|---|---|---|---|---|---|---|
| | brain | liver | heart | sk mus | colon | duodenum | ileum | jejunum | trachea | cava | bladder | ovary |
| miR-122a | 1 | 22996 | 0 | 6 | 57 | 1 | 0 | 24 | 0 | 52 | 3 | 0 |
| miR-128a | 9359 | 58 | 151 | 1039 | 181 | 100 | 177 | 123 | 74 | 222 | 158 | 126 |
| miR-128b | 6509 | 44 | 114 | 725 | 147 | 104 | 539 | 143 | 62 | 198 | 115 | 92 |
| miR-129 | 505 | 1 | 3 | 0 | 4 | 5 | 7 | 11 | 1 | 1 | 0 | 0 |
| miR-133a | 480 | 11 | 79791 | 243698 | 4375 | 1836 | 1793 | 2647 | 398 | 34508 | 7887 | 200 |
| miR-133b | 260 | 4 | 46148 | 132946 | 2861 | 850 | 746 | 1603 | 197 | 16954 | 3730 | 104 |
| miR-137 | 2186 | 0 | 2 | 0 | 101 | 37 | 38 | 91 | 0 | 0 | 0 | 5 |
| miR-301 | 113 | 3 | 46 | 0 | 30 | 20 | 32 | 3 | 2 | 26 | 25 | 5 |
| miR-302c | 92 | 0 | 8 | 0 | 0 | 0 | 0 | 0 | 40 | 14 | 0 | 0 |
| miR-31 | 97 | 0 | 0 | 0 | 33 | 678 | 965 | 237 | 235 | 35 | 146 | 3 |
| miR-323 | 678 | 6 | 7 | 5 | 26 | 26 | 20 | 54 | 6 | 16 | 11 | 24 |
| miR-330 | 146 | 2 | 7 | 1 | 6 | 6 | 8 | 6 | 3 | 8 | 7 | 2 |
| miR-346 | 166 | 3 | 7 | 3 | 5 | 5 | 4 | 5 | 3 | 6 | 2 | 3 |
| miR-34b | 37 | 0 | 1 | 0 | 4 | 3 | 6 | 5 | 302 | 3 | 2 | 0 |
| miR-34c | 439 | 1 | 16 | 11 | 63 | 67 | 76 | 73 | 6720 | 73 | 55 | 14 |
| miR-375 | 14 | 47 | 1 | 2 | 724 | 3001 | 1466 | 1848 | 1467 | 4 | 1 | 0 |
| miR-9* | 3798 | 0 | 16 | 0 | 15 | 38 | 29 | 58 | 6 | 19 | 0 | 0 |
| miR-124a | 30765 | 2 | 5 | 11 | 22 | 8 | 21 | 9 | 5 | 3 | 5 | 4 |
| miR-138 | 5049 | 3 | 8 | 7 | 153 | 66 | 96 | 98 | 31 | 16 | 5 | 5 |
| miR-149 | 1413 | 4 | 38 | 6 | 68 | 17 | 58 | 33 | 55 | 38 | 77 | 98 |
| miR-153 | 54212 | 62 | 602 | 268 | 2347 | 1133 | 964 | 2083 | 1179 | 450 | 1337 | 5958 |
| miR-181c | 764 | 6 | 36 | 13 | 87 | 48 | 56 | 74 | 38 | 54 | 48 | 92 |
| miR-18a | 167 | 69 | 51 | 16 | 179 | 187 | 82 | 364 | 77 | 227 | 113 | 114 |
| miR-205 | 17 | 7 | 3 | 11 | 12 | 6 | 10 | 6 | 9545 | 24 | 3198 | 16 |
| miR-216 | 0 | 0 | 0 | 0 | 0 | 85 | 0 | 0 | 0 | 0 | 0 | 0 |
| miR-217 | 14 | 3 | 3 | 1 | 5 | 645 | 4 | 5 | 10 | 15 | 10 | 5 |
| miR-338 | 348 | 4 | 11 | 1 | 23 | 31 | 34 | 12 | 6 | 8 | 20 | 4 |
| miR-383 | 87 | 0 | 0 | 0 | 6 | 1 | 2 | 1 | 0 | 0 | 1 | 44 |

TABLE 2B
Copies per cell in three human tissues and seven human cell
lines of 28 miRNAs determined using real time PCR analysis.

| | pancreas | testicle | kidney | HEPG2 | HUH1 | HELA | PERC6 | DLD1 | HCT116 | RKO |
|---|---|---|---|---|---|---|---|---|---|---|
| miR-122a | 1 | 15 | 72 | 4 | 3292 | 0 | 2 | 0 | 0 | 0 |
| miR-128a | 73 | 119 | 73 | 72 | 173 | 62 | 329 | 502 | 354 | 13 |
| miR-128b | 63 | 90 | 47 | 51 | 151 | 50 | 459 | 374 | 301 | 184 |
| miR-129 | 11 | 23 | 3 | 1 | 1 | 2 | 15 | 0 | 1 | 0 |
| miR-133a | 53 | 210 | 282 | 2 | 6 | 3 | 10 | 3 | 3 | 3 |
| miR-133b | 29 | 111 | 159 | 0 | 1 | 0 | 1 | 0 | 0 | 1 |
| miR-137 | 0 | 0 | 0 | 0 | 15 | 0 | 15 | 4 | 0 | 72 |
| miR-301 | 10 | 14 | 0 | 1 | 109 | 132 | 976 | 394 | 545 | 509 |
| miR-302c | 0 | 0 | 0 | 0 | 13 | 0 | 1217 | 88 | 208 | 561 |
| miR-31 | 7 | 0 | 148 | 0 | 0 | 670 | 430 | 140 | 5535 | 6381 |
| miR-323 | 20 | 24 | 9 | 1 | 1 | 1 | 5 | 1 | 3 | 1 |
| miR-330 | 3 | 2 | 3 | 2 | 6 | 14 | 19 | 27 | 19 | 42 |
| miR-346 | 1 | 3 | 3 | 5 | 10 | 9 | 23 | 5 | 22 | 7 |
| miR-34b | 1 | 486 | 1 | 0 | 0 | 0 | 19 | 0 | 1 | 0 |
| miR-34c | 11 | 10433 | 21 | 1 | 17 | 1 | 120 | 2 | 2 | 6 |
| miR-375 | 9228 | 34 | 5 | 1 | 3 | 0 | 3 | 123 | 37 | 1 |
| miR-9* | 0 | 0 | 0 | 0 | 0 | 0 | 20 | 0 | 229 | 0 |
| miR-124a | 2 | 21 | 25 | 2 | 2 | 1 | 9 | 1 | 2 | 3 |
| miR-138 | 7 | 8 | 69 | 2 | 2 | 1 | 19 | 3 | 2 | 1 |
| miR-149 | 18 | 79 | 20 | 12 | 83 | 30 | 164 | 74 | 158 | 50 |
| miR-153 | 1574 | 6223 | 255 | 18 | 37 | 47 | 205 | 47 | 1655 | 7182 |
| miR-181c | 21 | 49 | 18 | 11 | 33 | 16 | 67 | 9 | 33 | 98 |
| miR-18a | 60 | 280 | 143 | 104 | 1622 | 1419 | 11707 | 730 | 740 | 2351 |
| miR-205 | 9 | 1477 | 36 | 15 | 35 | 8 | 76 | 2 | 30 | 45 |
| miR-216 | 3035 | 0 | 0 | 0 | 0 | 0 | 0 | 0 | 0 | 0 |
| miR-217 | 6018 | 32 | 27 | 2 | 69 | 114 | 38 | 1 | 2 | 3 |
| miR-338 | 22 | 16 | 6 | 1 | 3 | 7 | 13 | 0 | 0 | 0 |
| miR-383 | 0 | 3 | 0 | 0 | 0 | 0 | 4 | 0 | 0 | 1 |

These data, and data not shown, allow the following miRNAs to be identified as tissue enriched:

miR-122a is expressed primarily in the liver;

miR-133 is expressed primarily in heart and skeletal muscle, and is also readily detectable in organs that contain smooth muscle tissue such as the intestine, vena cava, and bladder, and in addition, miR-1 and miR-206 are also expressed primarily in muscle;

miR196a, miR196b, miR-204, miR-10a and miR-10b are expressed primarily in the kidney;

miR-216, miR-217 and miR-375 are expressed primarily in the pancreas; and miR-124a, miR-9*; miR-9, miR-219, miR-137, miR-323, miR-330, miR-346, miR-153, miR-128a, miR-338, miR-7, miR-329, miR-132, miR-433, miR-128b, miR-138, miR-212, miR-340, miR-149, miR-181, miR-383, and miR-129 are expressed primarily in the brain.

### Example 2

### Pooled Sample Study: miR-122 and miR-133 Levels in Blood Plasma are Biomarkers for Tissue Injury to Liver and Muscle, Respectively

To explore the feasibility of detecting miRNA tissue injury biomarkers in plasma, a pilot study was performed in which pooled blood plasma samples from rat toxicology studies were analyzed using a qPCR-based assay (Raymond et at, 2005, RNA 11:1737; WO 2006/081282). The miRNAs miR-122 and miR-133 were chosen for quantification because they are extremely abundant and specific to liver and muscle, respectively (FIG. 1). Stored plasma samples from rats that had previously been treated with a liver damaging agent trichlorobromomethane (CBrCl₃) or with one of two muscle damaging agents 2,3,5,6-tetramethyl-p phenylenediamine (TMPD) or cerivastatin) were assayed form miRNA levels using qPCR.

Animals Male and female Sprague-Dawley rats were obtained from Charles River Laboratories, Inc. (Raleigh, N.C.). The rats were approximately seven to eight weeks of age and weighed from 125 to 325 grams at the start of the study. The animals were acclimated for approximately one week and randomized into treatment and control groups. During the toxicity studies targeting liver, the animals were maintained on a caloric-restricted diet, and fasted overnight prior to necropsy. Doses were different for each model compound tested and were calculated based on animal body weight. For the compounds, routes of administration, dose groups, and time points, see the below compound section.

Test Compounds

Skeletal Muscle Toxicant
2,3,5,6-tetramethyl-p phenylenediamine (TMPD)
Doses: 6 mg/kg bid
Dosing: 0.9% NaCl by subcutaneous injection
Animals: Sprague Dawley Male Rats
Time Points Day 3, TMPD Plasma (Day 3) and Controls (5 samples, 100 ul each pooled=500 ul total).

Liver Toxicant
Trichlorbromomethane (CCl₃Br)
Doses: 0.1 ml/kg
Dosing: 100% liquid paraffin by oral gavage
Animals: Sprague Dawley Male Rats
Time Point: Day 2, 4 CCl₃Br Plasma (Days 2 & 4) and Controls (5 samples, 100 ul each pooled=500 ul total)

Liver and Muscle Toxicant
Cerivastatin
Doses: 1.0 mg/kg/day
Dosing: 0.5% methylcellulose by oral gavage
Animals: Sprague Dawley Female Rats
Time Point Day 14 Cerivastatin Plasma (Day 14) and Controls (5 samples, 100 ul each pooled=500 μl total)

Plasma Samples All plasma samples were collected in EDTA tubes at final necropsy from the vena cava. Samples were inverted several times, and centrifuged for 20 minutes at 2,900 RPM at 4° C. Plasma samples were removed and stored at −70° C. Pooled blood plasma samples from rat toxicology studies were analyzed using a qPCR-based assay (Raymond et al, 2005, RNA 11:1737; WO 2006/081282). Control slope and intercept values for miR-122 and miR-133 were calculated from a concentration series of each microRNA added to total yeast RNA and then analyzed using the qPCR assay for each miRNA. The slope and intercept values from the standard curve were used to calculate the amount of each respective miRNA in each sample expressed as the number of miRNA copies per 20 ng of total RNA in each sample. Cycle threshold values (Ct) for each sample were converted to miRNA copy numbers by comparison to standard curves generated using single stranded mature miRNAs and are expressed as copies/20 pg input RNA (approximately equivalent to copies/cell).

Table 3 shows the amount of miR-122 in the pooled plasma samples from treated and non-treated animals as compared to miR-122 levels in human liver tissue, human skeletal muscle tissue and rat liver tissue.

TABLE 3
miR-122 Levels - Pooled Samples

| | | Ct | | Sample, | |
|---|---|---|---|---|---|
| | Ct | Standard | | Total RNA | Copies/20 |
| | Mean | Deviation | Copies | Input (ng) | pg |
| Human | 12.5589 | 0.21999 | 9.7E+08 | 250 | 77575.3 |
| Liver | | | | | |
| Skeletal | 28.6093 | 0.17431 | 17988 | 250 | 1.4 |
| Muscle | | | | | |
| Rat Liver | 12.9572 | 0.21206 | 7.4E+08 | 250 | 59199.7 |
| Cerivastatin | 27.6892 | 0.41695 | 33591.7 | 250 | 2.7 |
| control | | | | | |
| Cerivastatin | 24.2231 | 0.28943 | 353217 | 250 | 28.3 |
| CBrCl3 | 26.2392 | 0.35099 | 89881.9 | 250 | 7.2 |
| Control | | | | | |
| CBrCl3 | 12.6961 | 0.18656 | 8.8E+08 | 250 | 70674.9 |
| TMPD | 24.5028 | 0.21782 | 292127 | 250 | 23.4 |
| Control | | | | | |
| TMPD | 26.2479 | 0.29099 | 89353.3 | 250 | 7.1 |

Table 4 shows the amount of miR-133 in the pooled plasma samples from treated and non-treated animals as compared to miR-122 levels in human liver tissue, human skeletal muscle tissue and rat liver tissue.

TABLE 4
miRNA-133 Levels - Pooled Samples

| | | | | Sample, | |
|---|---|---|---|---|---|
| | | Ct | | Total RNA | Copies/20 |
| | Ct Mean | StdDev | Copies | Input (ng) | pg |
| Human | 30.9357 | 0.36411 | 35174.3 | 250 | 2.8 |
| Liver | | | | | |
| Skeletal | 16.6732 | 0.19486 | 5.8E+08 | 250 | 46574.9 |
| Muscle | | | | | |
| Rat Liver | 31.2206 | 0.41725 | 28969.3 | 250 | 2.3 |
| Cerivastatin | 34.1014 | 0.76853 | 4072.02 | 250 | 0.3 |
| control | | | | | |
| Cerivastatin | 23.1065 | 0.24999 | 7279643 | 250 | 582.4 |
| CBrCl3 | 31.7688 | 0.35122 | 19943.1 | 250 | 1.6 |
| Control | | | | | |
| CBrCl3 | 29.6296 | 0.21686 | 85617.9 | 250 | 6.8 |
| TMPD | 34.8212 | 0.76844 | 2494 | 250 | 0.2 |
| Control | | | | | |
| TMPD | 23.7323 | 0.23513 | 4753175 | 250 | 380.3 |

The data presented in Table 3 and Table 4 shows miR-122 was detected at very high levels in the plasma of liver-damaged animals, but not in the muscle-damaged animals or the controls. Conversely, miR-133 was detected at very high levels in the plasma of muscle-damaged animals, but not in the liver-damaged animals or the controls. These results showed that miRNAs could not only be successfully detected in blood plasma, but that miR-122 and miR-133 appear to be specific biomarkers of tissue injury to liver and muscle, respectively.

### Example 3

### Individual Sample Study: miR-122 and miR-133 Levels in Blood Plasma are Biomarkers for Tissue Injury to Liver and Muscle, Respectively

This examples confirms the usefulness of the tissue-specific miRNAs miR-122 and miR-133 as biomarkers for tissue injury to liver and muscle, respectively. Analysis of plasma from a limited number of rat models of tissue injury shows that miR-122 has good sensitivity and specificity as compared to the ALT/AST assays that are the accepted standards of liver toxicology.

To follow up on the initial results shown in Example 2, blood samples from 44 individual rats that had been treated with varying doses of CBrCl₃, CCl₄ (another liver toxicant), or TMPD were evaluated using qPCR of miR-122 and miR-133. In addition, data from histopathology and a panel of standard biomarkers (ALT, AST, CK, skTnI, etc.) were also available for these same individual rats and were used to compare the results from microRNA assays with the standard histopathology and protein biomarkers.

Animals Male and female Sprague-Dawley rats were obtained from Charles River Laboratories, Inc. (Raleigh, N.C.). The rats were approximately seven to eight weeks of age and weighed from 125 to 325 grams at the start of the study. The animals were acclimated for approximately one week and randomized into treatment and control groups. During the toxicity studies targeting liver, the animals were maintained on a caloric-restricted diet, and fasted overnight prior to necropsy. Doses were different for each model compound tested and were calculated based on animal body weight. For the compounds, routes of administration, dose groups, and time points, see the below compound section.

Test Compounds

Skeletal Muscle Toxicant
2,3,5,6-tetramethyl-p phenylenediamine (TMPD)
Doses: 2, 4 mg/kg bid
Dosing: 0.9% NaCl by subcutaneous injection
Animals: Sprague Dawley Male Rats
Time Points: TMPD Plasma (Day 2) and Controls (5 samples per group, 1000 each pooled=500 μl total).

Liver Toxicant
Trichlorobromomethane (CCl₃Br)
Doses: 0.03, 0.1 ml/kg
Dosing: 100% liquid paraffin by oral gavage
Animals: Sprague Dawley Male Rats
Time Point: CCl₃Br Plasma (Days 2 & 4) and Controls (4 controls, 3 samples per group, 100 μl each)
Carbon Tetrachloride (CCl₄)
Doses: 0.03, 0.1, 0.3 ml/kg
Dosing: 100% liquid paraffin by oral gavage
Animals: Sprague Dawley Male Rats
Time Point: CCl₄ Plasma (Days 2) and Controls (4 samples per group, 100 μl each)

Plasma Samples All plasma samples were collected in EDTA tubes at final necropsy from the vena cava. Samples were inverted several times, and centrifuged for 20 minutes at 2,900 RPM at 4° C. Plasma samples were removed and stored at −70° C. Pooled blood plasma samples from rat toxicology studies were analyzed using a qPCR-based assay (Raymond et al, 2005, RNA 11:1737; WO 2006/081282). Control slope and intercept values for miR-122 and miR-133 were calculated from a concentration series of each microRNA added to total yeast RNA and then analyzed using the qPCR assay for each miRNA. The slope and intercept values from the standard curve were used to calculate the amount of each respective miRNA in each sample expressed as the number of miRNA copies per 20 ng of total RNA in each sample. Cycle threshold values (Ct) for each sample were converted to miRNA copy numbers by comparison to standard curves generated using single stranded mature miRNAs and are expressed in Table 4 as copies/20 pg input RNA (approximately equivalent to copies/cell).

TABLE 4
MicroRNA and clinical chemistry measurements in blood plasma samples
and corresponding liver or muscle histopatholgy scores.

| Treatment | | | miR- | miR- | AST | ALT | Liver Histopathology Scores* | | | |
|---|---|---|---|---|---|---|---|---|---|---|
| CCl₄ | | Animal | 122 | 133 | Unit | Unit | Necrosis | Degeneration | Inflammation | Vacuolation |
| control | | 06-2712 | 0.5 | 0.5 | 94 | 22 | 0 | 0 | 0 | 0 |
| | | 06-2714 | 1.4 | 0.5 | 86 | 25 | 0 | 0 | 0 | 0 |
| | | 06-2716 | 2.6 | 0.7 | 73 | 22 | 0 | 0 | 0 | 0 |
| | | 06-2718 | 5.6 | 1.1 | 153 | 23 | 0 | 0 | 0 | 0 |
| 0.03 | mL/kd | 06-2750 | 2.4 | 0.3 | 92 | 25 | 0 | 0 | 0 | 0 |
| | | 06-2752 | 5.5 | 0.5 | 88 | 20 | 0 | 0 | 0 | 0 |
| | | 06-2754 | 8.7 | 0.3 | 76 | 25 | 0 | 0 | 0 | 0 |
| | | 06-2756 | 10.0 | 1.0 | 84 | 25 | 0 | 0 | 0 | 0 |
| 0.1 | mL/kg | 06-2770 | 7.9 | 0.3 | 96 | 25 | 0 | 0 | 0 | 0 |
| | | 06-2772 | 107.4 | 0.3 | 116 | 40 | 1 | 1 | 2 | 1 |
| | | 06-2774 | 14.3 | 0.4 | 102 | 40 | 1 | 1 | 1 | 1 |
| | | 06-2776 | 119.4 | 0.4 | 126 | 41 | 0 | 0 | 0 | 1 |
| 0.3 | mL/kg | 06-2790 | 1563.6 | 1.4 | 345 | 87 | 1 | 2 | 2 | 0 |
| | | 06-2792 | 194.3 | 0.4 | 216 | 77 | 1 | 2 | 1 | 0 |
| | | 06-2794 | 191.4 | 0.3 | 157 | 62 | 1 | 2 | 2 | 0 |
| | | 06-2798 | 164.6 | 0.1 | 225 | 56 | 1 | 2 | 2 | 0 |

| | | miR- | miR- | AST | ALT | Liver Histopathology Scores | | | |
|---|---|---|---|---|---|---|---|---|---|
| CBrCl₃ | | 122 | 133 | Unit | Unit | Necrosis | Degeneration | Inflammation | Mitotic Index |
| control | 06-2720 | 5.5 | 0.5 | 83 | 26 | 0 | 0 | 0 | 0 |
| | 06-2722 | 4.1 | 0.6 | 105 | 26 | 0 | 0 | 0 | 0 |
| | 06-2724 | 3.1 | 1.2 | 99 | 26 | 0 | 0 | 0 | 0 |
| | 06-2728 | 15.9 | 0.2 | 97 | 24 | 0 | 0 | 0 | 0 |
| 0.03 mL/kg | 06-2832 | 860.8 | 0.7 | 366 | 142 | 1 | 2 | 1 | 0 |
| Day 2 | 06-2834 | 45.1 | 0.2 | 83 | 36 | 0 | 0 | 0 | 0 |
| | 06-2838 | 47.3 | 0.4 | 93 | 36 | 0 | 1 | 1 | 0 |
| 0.03 mL/kg | 06-2840 | 90.2 | 1.2 | 138 | 34 | 0 | 0 | 0 | 0 |
| Day 4 | 06-2842 | 56.2 | 2.0 | 118 | 43 | 0 | 1 | 0 | 0 |
| | 06-2844 | 20.5 | 0.3 | 108 | 32 | 0 | 1 | 0 | 0 |
| 0.1 mL/kg | 06-2860 | 4049.2 | 0.8 | 1543 | 1388 | 3 | 2 | 2 | 1 |
| Day 4 | 06-2862 | 335.5 | 0.4 | 370 | 191 | 1 | 1 | 2 | 1 |
| | 06-2864 | 304.0 | 0.4 | 519 | 223 | 1 | 1 | 2 | 2 |

Muscle
Histopathology

| | | | | | | | Quad | Soleus |
|---|---|---|---|---|---|---|---|---|
| | | miR- | miR- | AST | ALT | CK | (Overall | (Overall |
| TMPD | | 122 | 133 | U/L | U/L | U/L | Damage) | Damage) |
| control | 05-1640 | 2.7 | 0.7 | 97 | 27 | 567 | 0 | 0 |
| | 05-1642 | 1.3 | 0.8 | 97 | 23 | 579 | 0 | 0 |
| | 05-1644 | 2.7 | 1.8 | 84 | 35 | 368 | 0 | 0 |
| | 05-1646 | 6.9 | 0.3 | 107 | 29 | 612 | 0 | 0 |
| | 05-1648 | 6.8 | 0.4 | 88 | 33 | 362 | 0 | 0 |
| 2 mg/kg | 05-1670 | 2.6 | 0.9 | 120 | 29 | 424 | 0 | 0 |
| bid | 05-1672 | 5.6 | 4.3 | 164 | 36 | 695 | 0 | 0 |
| | 05-1674 | 7.3 | 9.3 | 203 | 37 | 696 | 1 | 0 |
| | 05-1676 | 0.8 | 1.3 | 111 | 27 | 450 | 0 | 0 |
| | 05-1678 | 2.2 | 4.1 | 167 | 30 | 463 | 0 | 0 |
| 4 mg/kg | 05-1700 | 9.5 | 19.3 | 357 | 60 | 363 | 1 | 1 |
| bid | 05-1702 | 1.8 | 63.9 | 1250 | 133 | 767 | 1 | 1 |
| | 05-1704 | 15.7 | 79.3 | 1740 | 215 | 1252 | 2 | 0 |
| | 05-1706 | 11.7 | 63.8 | 700 | 94 | 439 | 1 | 1 |
| | 05-1708 | 3.2 | 16.7 | 501 | 56 | 343 | 2 | 0 |

*Histopathology scores: 1 = very slight, 2 = slight or small, 3 = moderate, 4 = marked, 5 = severe tissue injury.

The data in Table 4 show that miR-122 signal was detected in all cases where liver histopathology was detected. Of special interest are animals #06-2834 and #06-2840, where, although no liver histopathology was detected, high levels of miR-122 in these animals (relative to controls) are consistent with their liver toxicant treatment and the liver histopathology in their cohorts. This data suggests that liver injury may have occurred but was not observed by the pathologist in the section examined.

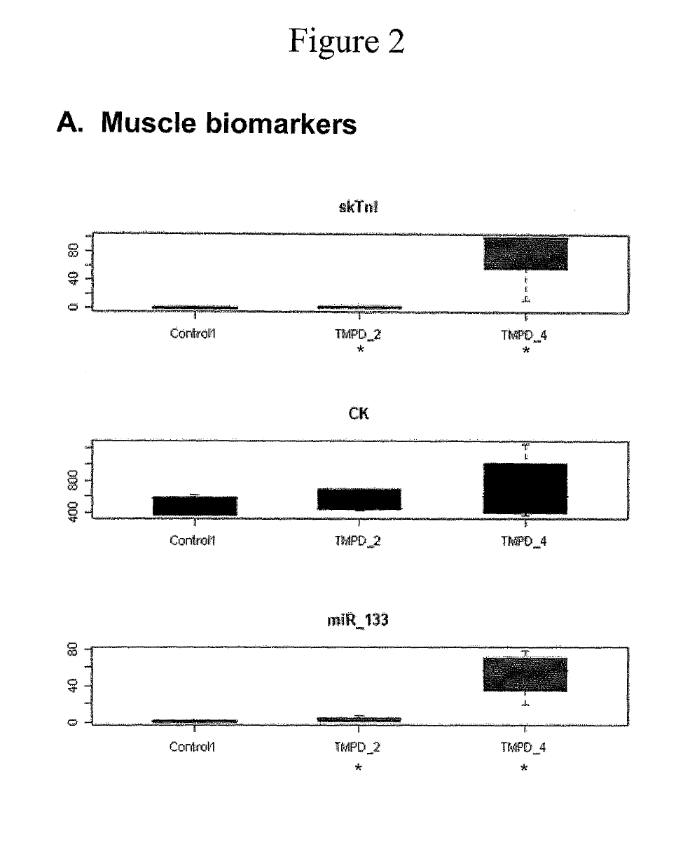

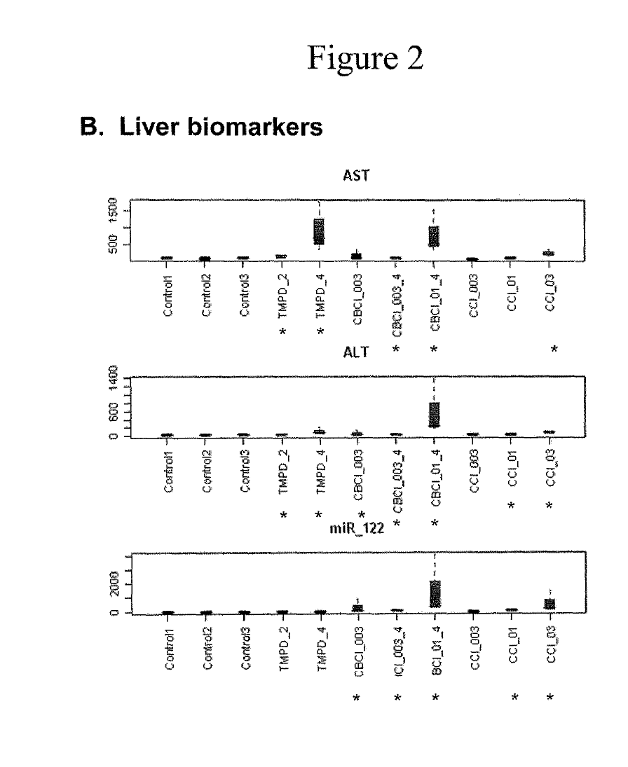

FIG. 2 shows box plots of a subset of the miRNA measurements displayed in Tables 4 and 5 and standard biomarker levels in plasma from dosed rats. Measurements taken from individual dosed rats are summarized in boxplots, where the first and third quartile are indicated by the borders of the rectangle. Measurements that are significantly greater (p<0.05, Mann-Whitney) than the controls are shown with an asteric under the sample name. Panel A) shows measurement of muscle biomarkers: skeletal troponin (skTnl), creatine kinase (CK), and miR-133 (miR 133). Panel B) shows measurement of liver injury biomarkers: aspartate aminotransferase (AST), alanine aminotransferase (ALT), and miR-122 (miR_{—}122). Control1: control group for TMPD treatments. TMPD_{—}2: 2 mg/kg TMPD dose, 2 days. TMPD_{—}4: 4 mg/kg TMPD dose, 2 days. Control2: control group for CBrCl₃ treatments. CBCl3_{—}003: 0.03 ml/kg CBrCl₃ dose, 2 days. CBCl3_{—}0034: 0.03 ml/kg CBrCl₃ dose, 4 days. CBCl3_{—}01_{—}4: 0.1 ml/kg CBrCl₃ dose, 4 days. Control3: control group for CCl₄ treatments. CCl_{—}003: 0.03 ml/kg CCl₄ dose, 2 days. CCl_{—}01: 0.1 ml/kg CCl₄ dose, 2 days. CCl_{—}03: 0.3 ml/kg CCl₄ dose, 2 days.

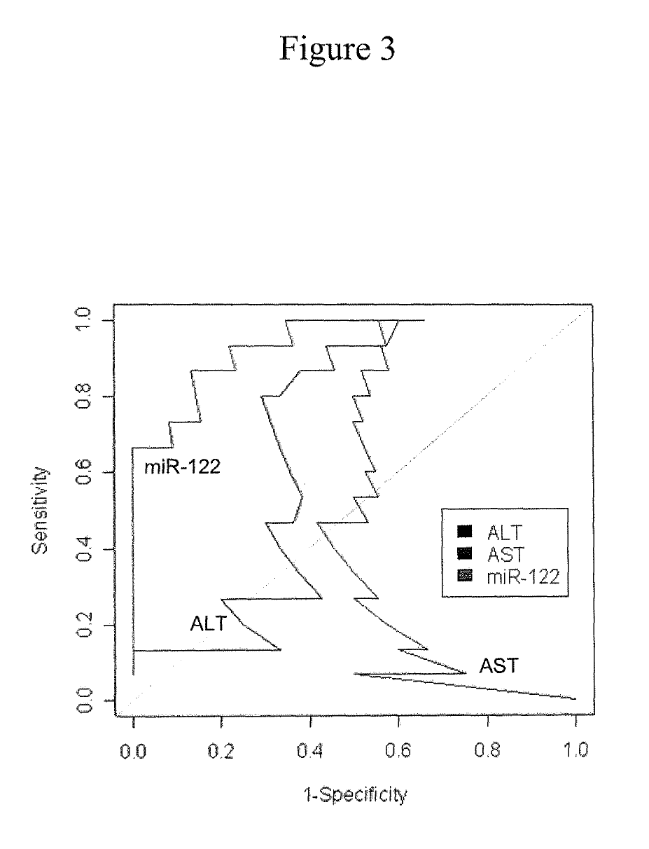

The data presented in Table 4 and FIG. 2 (control vs. toxicant-treated rats) shows that microRNAs compare favorably with the standard biomarkers for each tissue injury. For example, FIG. 2(A) demonstrates that miR-133 measurements can detect TMPD dosing in rats as sensitively as skeletal troponin levels and more sensitively than the creatine kinase enzymatic assay. Similarly, the data in FIG. 3 show that miR-122 measurements are reporting on CBrCl₃ and CCl₄ dosing as sensitively as alanine aminotransferase, and more sensitively than aspartate aminotransferase.

Furthermore, whereas ALT and AST were significantly elevated upon dosing with the muscle toxicant TMPD, miR-122 was not. Thus, miR-122 levels may be a more specific marker of liver injury than ALT and AST, given that no liver histopathology has been observed in TMPD-treated rats. In fact, as presented in FIG. 3, miR-122 is shown to be a better predictor of liver injury than ALT and AST using liver histopathology data as the reference in a receiver operating characteristic (ROC) curve analysis (Fawcett, 2006, Pattern Recognition Letters 27:861).

The ROC curve analysis data may be underestimating miR-122's predictive ability, however, as miR-122 may turn out to be more sensitive than liver histopathology observations in certain situations.

### Example 4

### MicroRNA miR-133 is a Blood Biomarker for Skeletal Muscle Injury in Dogs

This examples shows that miR-133 is a blood bourn biomarker for exercise induced tissue injury to muscle in dogs. Analysis of plasma from the dogs shows that miR-122 has good sensitivity and specificity as compared to the creatine kinase (CK), aspirate aminotransferase (AST) and alanine amino transferase (ALT) assays that are the accepted standards of myopathy.

Skeletal muscle responses to an exercise stimulus are complex. The muscle responds in the short term by increasing ATP production to meet the energy demands of the exercise. However, exercise can also elicit a longer term response in which the muscle adapts to future work requirements through hypertrophy and increasing oxidative metabolic capacity. These are major components of the classical training response. Additionally, exercise can result in muscle injury characterized by sarcolemmal damage, with or without evidence of inflammation and pain. The skeletal muscle responses evoked are dependent on the intensity, duration and type (eccentric, concentric) of exercise.

Alaskan sled dogs used in competition are elite athletes that undergo regular exercise training. The present study utilized Alaskan sled dogs before and after a prolonged endurance exercise training session to characterize the exercise-induced, time dependent changes in skeletal muscle gene expression.

In this study the test subjects were 14 Alaskan sled dogs who were randomly assigned to two groups—one group of 10 dogs who ran 160 km in 24 hours. All dogs completed the exercise regimen without evidence of muscle soreness. The second group consisted of 4 dogs that did not run. Blood samples were collected from all dogs within a 10 minute window at 2, 6, 12, and 24 hours after completing the run. RNA was extracted from blood samples and subjected to mircroRNA analysis using qPCR probes directed to miR-133. In addition, conventional plasma markers (CK, ALT, AST) were measured for all plasma samples to evaluate if the animals exhibit symptoms of exercise induced myopathy.

Plasma protein markers and miR-133 levels in the sled dogs over the pre- and post-run intervals are shown in Table 5.

TABLE 5
Pre- and post- exercise plasma biomarker values.

| | Before exercise | | | | Two hours post-exercise | | | |
|---|---|---|---|---|---|---|---|---|
| | miR133 | | | | miR133 | | | |
| | (copies/ | CK | ALT | AST | (copies/ | CK | ALT | AST |
| Dog | 20 pg) | U/L | U/L | U/L | 20 pg) | U/L | U/L | U/L |

Non-Runners

| | | | | | | | | |
|---|---|---|---|---|---|---|---|---|
| Carrot | 0.8 | 74 | 41 | 20 | 169.2 | 629 | 53 | 56 |
| Charger | 0.9 | 69 | 69 | 26 | 10.1 | 195 | 62 | 44 |
| Mr. | 7.1 | 82 | 60 | 19 | 8 | 212 | 51 | 25 |
| Freeze | | | | | | | | |
| Mary | 1.5 | 85 | 47 | 20 | 10.6 | 270 | 49 | 37 |

Runners

| Crunch | 0.7 | 103 | 57 | 29 | 1954 | 8042 | 102 | 342 |
|---|---|---|---|---|---|---|---|---|
| Texaco | 1.5 | 75 | 44 | 22 | 224 | 816 | 51 | 83 |
| Marge | 65.4 | 232 | 48 | 22 | 220.6 | 1488 | 75 | 112 |
| Cocoa | 1.5 | 83 | 46 | 28 | 1130.8 | 5185 | 76 | 272 |
| Gypsy | 2.6 | 90 | 36 | 26 | 162.8 | 1374 | 49 | 120 |
| Friskie | 2.2 | 95 | 62 | 25 | 1311.2 | 6740 | 91 | 359 |
| Gasso | 1.8 | 89 | 96 | 34 | 419.4 | 2847 | 101 | 165 |
| Ranger | 44.4 | 134 | 69 | 23 | 3249 | 7600 | 216 | 593 |
| Barley | 2.1 | 110 | 79 | 34 | 230.2 | 1427 | 102 | 128 |
| Poke | 2.5 | 105 | 70 | 25 | 127.5 | 1063 | 72 | 95 |
| | Six hours post-exercise | | | | 12 hours post-exercise | | | |
| | miR133 | | | | miR133 | | | |
| | (copies/ | CK | ALT | AST | (copies/ | CK | ALT | AST |
| Dog | 20 pg) | U/L | U/L | U/L | 20 pg) | U/L | U/L | U/L |

Non-Runners

| | | | | | | | | |
|---|---|---|---|---|---|---|---|---|
| Carrot | 10.1 | 245 | 46 | 39 | 5.4 | 173 | 42 | 32 |
| Charger | 3.2 | 158 | 58 | 40 | 10.3 | 116 | 54 | 34 |
| Mr. | 8.8 | 204 | 58 | 28 | 4.9 | 154 | 59 | 24 |
| Freeze | | | | | | | | |
| Mary | 1.3 | 183 | 50 | 35 | 1.7 | 143 | 45 | 26 |

Runners

| | | | | | | | | |
|---|---|---|---|---|---|---|---|---|
| Crunch | 175.9 | 5240 | 123 | 302 | 119.8 | 2740 | 115 | 234 |
| Texaco | 34.1 | 682 | 51 | 80 | 30.7 | 462 | 59 | 72 |
| Marge | 50.5 | 1231 | 80 | 102 | 48.6 | 861 | 94 | 97 |
| Cocoa | 190.6 | 3051 | 82 | 223 | 114.4 | 1528 | 87 | 174 |
| Gypsy | 25.6 | 1297 | 52 | 104 | 7.6 | 648 | 48 | 79 |
| Friskie | 199.6 | 4462 | 108 | 325 | 12.3 | 2724 | 112 | 237 |
| Gasso | 156.3 | 3039 | 116 | 182 | 31.7 | 1565 | 113 | 138 |
| Ranger | 793.6 | 9570 | 261 | 550 | 418.4 | 7276 | 233 | 390 |
| Barley | 28.4 | 1260 | 109 | 122 | 36.1 | 673 | 98 | 88 |
| Poke | 33.4 | 616 | 77 | 89 | 10.0 | 334 | 71 | 66 |

24 hours post-exercise

| | | | | |
|---|---|---|---|---|
| | miR133 | | | |
| | (copies/ | CK | ALT | AST |
| Dog | 20 pg) | U/L | U/L | U/L |

Non-Runners

| | | | | |
|---|---|---|---|---|
| Carrot | 1.1 | 157 | 54 | 33 |
| Charger | 1.5 | 96 | 69 | 40 |
| Mr. | 7.1 | 122 | 66 | 26 |
| Freeze | | | | |
| Mary | 1.8 | 128 | 59 | 34 |

Runners

| | | | | |
|---|---|---|---|---|
| Crunch | 57.1 | 1357 | 141 | 185 |
| Texaco | 10.9 | 242 | 78 | 64 |
| Marge | 5.5 | 458 | 95 | 69 |
| Cocoa | 13.2 | 512 | 96 | 109 |
| Gypsy | 0.9 | 228 | 49 | 57 |
| Friskie | 10.6 | 584 | 107 | 104 |
| Gasso | 7.0 | 503 | 117 | 88 |
| Ranger | 69.1 | 2183 | 232 | 220 |
| Barley | 2.5 | 247 | 101 | 57 |
| Poke | 1.7 | 109 | 61 | 30 |

Clear indication of exertion induced tissue injury to muscle was obtained based upon the levels of the traditional protein biomarkers. The peaks in AST and CK levels at the two hour post-exercise time intervals are indicators of exercise induced tissue injury to muscle. The AST and CK levels were also highly correlated with miR-133 levels that also peaked in the two-hour post exercise plasma samples. However, unlike miR-133, which is specific to muscle cells, the ALT, AST, and to a lesser extent CK values are not tissue specific and also can arise from injury to other tissues, such as heart (CK, AST, ALT) or liver (ALT, AST). By 24 hours after exercise the blood plasma levels of CK, AST, ALT and miR-133 all dropped to almost pre-exercise levels.

### Example 5

### MicroRNA miR-124 is a Blood Biomarker for Ischemic Tissue Injury in Brain

This examples shows that miR-124 is a biomarker for tissue injury in brain. Analysis of blood plasma from a limited number of rat models of stroke induced tissue injury shows that miR-124 has good sensitivity and specificity as a tissue injury biomarker in brain.

A rat stroke model was used to assess the presence of miR-124 in blood plasma as a function of time. Ischemia was induced in femoral vein cannulated male Sprague-Dawley rats (Charles-River) using the middle cerebral artery occlusion (MCAO) intra-luminal filament method (Longa et al., 1989, Stroke 20:84-91). Both transient (t) and permanent (p) and MCAO methods were used to obtain different degrees of tissue injury in the brain. Animals with sham operations were included as controls (sham). Blood (EDTA plasma) was collected at 2 hours [sham (n=4), tMCAO (n=3), pMCAO (n=5)] and 24 hours [sham (n=4), pMCAO (n=4), tMCAO (n=4)] post-surgery. Total RNA was obtained and qPCR was performed as described in Examples 2 and 3. Surgical induced stroke treatment and miRNA measurement data are presented in Table 6.

TABLE 6
miR-124 qPCR measurements in rats
receiving surgery induced stroke.

| 2 hours post-surgery | | | 24 hours post-surgery | | |
|---|---|---|---|---|---|
| | | miR-124a | | | miR-124a |
| | | copies/ | | | copies/ |
| Animal | Treatment | 20 pg | Animal | Treatment | 20 pg |
| RS_CC5 | Sham | 0.01 | RS_CC3 | Sham | 0.03 |
| RS_BC6 | Sham | 0.04 | RS_BC1 | Sham | 0.02 |
| RS_RS5 | Sham | 0.09 | RS_RS2 | Sham | 0.07 |
| RS_CF6 | Sham | 0.06 | RS_CF3 | Sham | 0.04 |
| RS_CC6 | Trans | 2.17 | RS_CC1 | Trans | 1.31 |
| RS_BC2 | Trans | 0.14 | RS_RS4 | Trans | 0.57 |
| RS_CF5 | Trans | 0.22 | RS_CF1 | Trans | 0.39 |
| RS_CC2 | Perm | 0.12 | RS_CC4 | Trans | 0.81 |
| RS_RS3 | Perm | 0.07 | RS_BC3 | Trans | 3.82 |
| RS_RS6 | Perm | 0.08 | RS_BC4 | Perm | 0.81 |
| RS_CF4 | Perm | 0.07 | RS_RS1 | Perm | 0.86 |
| RS_BC5 | Perm | 0.12 | RS_CF2 | Perm | 2.29 |

The miRNA measurement data presented in Table 6, shows that at about 2-hours post-surgery in the tMCAO group miRNA-124 levels in blood plasma begins to slightly increase compared to sham-operated rats. At 24 hours post-surgery, there is an about 19-33 fold increase in miR-124 in both t- and pMCAO rats when compared to sham-operated rats. Sham-operated rats showed consistently low (basal) levels of miRNA-124 throughout the 24 hours post-surgery. There were no differences in plasma miRNA-124 levels between t- and p-MCAO operated animals.

### Example 6

### MicroRNA miR-10a Levels in Urine is a Biomarker for Tissue Injury to Kidney

This examples shows the usefulness of the tissue-specific miRNA miR-10a as a biomarkers for tissue injury to kidney. Analysis of urine from a limited number of rat models of compound-induced tissue injury kidney shows that miR-10a has sensitivity and specificity as compared to the histopathology of kidney tissue that is the accepted standards of kidney toxicology.

Urine samples from five individual rats that had been treated with a single dose of cisplatin (7 mg/kg), and nine individual rats that had been treated with a single dose of gentamicin (240 mg/kg) were evaluated using OCR of miR-10a. In addition, data from histopathology was also available for these same individual rats and were used to compare the results from microRNA assays with the standard histopathology scores of tissue injury in kidney.

Animals Male and female Sprague-Dawley rats were obtained from Charles River Laboratories, Inc. (Raleigh, N.C.). The rats were approximately seven to eight weeks of age and weighed from 125 to 325 grams at the start of the study. The animals were acclimated for approximately one week and randomized into treatment and control groups. During the toxicity studies targeting kidney, the animals were maintained on a caloric-restricted diet, and fasted overnight prior to necropsy. Doses were different for each model compound tested and were calculated based on animal body weight. For the compounds, routes of administration, dose groups, and time points, see the below Test Compound section.

Test Compounds

Kidney Toxicants
Cisplatin
Doses: 7.0 mg/kg
Dosing: 0.9% NaCl by intraperitoneal injection
Animals: Sprague Dawley Male Rats
Time Point Day 8
Gentamicin
Doses: 20, 80,240 mg/kg/day
Dosing: 0.9% NaCl by intraperitoneal injection
Animals: Sprague Dawley Male Rats
Time Point Day 3, 9, 12

Urine Samples Urine samples were collected overnight on either dry or wet ice, aliquoted, and stored at −70° C. Urine samples were removed and stored at −70° C. Urine samples were thawed on ice and about 1 milliliter (ml) of each sample placed into a tube. Each sample was placed in a room temperature speed-vac to reduce sample volume to about 200 microliters (μl). About 1 ml of trizol was added to the viscous concentrated urine solution and RNA extracted using Ambion BlycoBlue and alcohol precipitation.

Samples from rat toxicology studies were analyzed for miRNA levels using a qPCR-based assay (Raymond et al, 2005, RNA 11:1737; WO 2006/081282). Control slope and intercept values for miR-10a was calculated from a concentration series of miR-10a added to total yeast RNA and then analyzed using the qPCR assay. The slope and intercept values from the standard curve were used to calculate the amount of miR-10a in each sample expressed as the number of miRNA copies per 20 ng of total RNA in each sample. Cycle threshold values (Ct) for each sample were converted to miRNA copy numbers by comparison to a standard curve generated using single stranded mature miR-10a and are expressed in Table 7 as copies/20 pg input RNA (approximately equivalent to copies/cell).

Histopathology of kidney tissues was evaluated at day 8 for the cisplatin treated animals and at day 12 for the animals treated with gentamicin. The score of 5 indicates that there was severe tissue injury to the proximal tublule in each animal by the final day of each compound treatment.

TABLE 7
MicroRNA and histopathology measurements in urine samples
and corresponding histopatholgy scores.

| Treatment | Animal | miR-10a | Kidney Histopathology |
|---|---|---|---|
| Cisplatin | | | Proximal tubule overall |
| | | | tissue injury |
| | | | (highest severity grade |
| | | | measured at Day 8) |
| control | 04-0418 | <0.1 | 0 |
| | 04-0420 | <0.1 | 0 |
| 7 mg/kg | 04-0472 | 27.68 | 5 |
| Day 8 | 04-0474 | 41.80 | 5 |
| | 04-0478 | 74.54 | 5 |
| | 04-0486 | 120.44 | 5 |
| | 04-0496 | 108.53 | 5 |
| Gentamicin | | | Proximal tubule overall |
| | | | tissue injury |
| | | | (highest severity grade |
| | | | measured at Day 12) |
| Control | | | |
| Day 3 | 04-514 | <0.1 | 0 |
| Day 9 | 04-516 | 0.16 | 0 |
| Day 9 | 04-522 | <0.1 | 0 |
| Day 9 | 04-524 | 0.05 | 0 |
| Day 9 | 04-530 | 0.13 | 0 |
| 240 mg/kg/ | 04-612 | 11.98 | |
| day | | | |
| Day 3 | 04-614 | 3.51 | |
| | 04-618 | 0.03 | |
| | 04-622 | 0.05 | |
| | 04-624 | 10.00 | |
| 240 mg/kg/ | 04-612 | 0.96 | |
| day | | | |
| Day 9 | 04-614 | 0.64 | |
| | 04-616 | 16.88 | |
| | 04-618 | 51.03 | |
| | 04-620 | 6.61 | |
| | 04-622 | 4.90 | |
| | 04-624 | 58.70 | |
| | 04-626 | 38.01 | |
| | 04-630 | 25.61 | |
| 240 mg/kg/ | 04-612 | 3.5 | 5 |
| day | | | |
| Day 12 | 04-614 | 9.46 | 5 |
| | 04-616 | 4.17 | 5 |
| | 04-618 | 2.13 | 5 |
| | 04-620 | 4.99 | 5 |
| | 04-622 | 3.46 | 5 |
| | 04-624 | 3.86 | 5 |
| | 04-626 | 23.75 | 5 |
| | 04-630 | 10.40 | 5 |

The data presented in Table 7 (control vs. compound-treated rats) shows that miR-10a levels are correlated with compound induced kidney tissue damage. For example, the data in Table 7 demonstrates that miR-10a measurements can detect tissue injury in kidney associated with cisplatin and gentamicin dosing in rats.

### Example 7

### MicroRNA miR-122 is a Plasma Biomarker for Human Hepatitis C Virus (HCV) Infection of Liver

This example shows the usefulness of the liver-specific miRNA miR-122 as a biomarker for human HCV infection. As shown above in Examples 2 and 3, analysis of plasma from number of rat models of compound-induced tissue injury of liver shows that miR-122 has sensitivity and specificity as compared to the histopathology of liver tissue that is the accepted standards of liver toxicology. Chronic HCV infection can induce liver fibrosis and cirrhosis. The experiment present in this Example 7, was performed to determine whether increased plasma miR-122 levels are observed in humans individuals chronically infected with HCV as a possible non-invasive biomarker for HCV-induced liver damage.

Plasma samples from subjects chronically infected with HCV of either genotype 1a or 1b and uninfected controls were obtained from BocaBiolistics, LLC (Coconut Creek, Fla.). All chronically infected HCV subjects were naïve to HCV therapy. Genotyping of HCV is helpful in assessing the likelihood of response to therapy. Patients infected with genotype 1 HCV have much lower rates of response to interferon based treatment than do patients with genotype 2 or 3. Copy number of miR-122 was determined as previously described in Example 3, and the resulting values are listed in Table 8, which additionally shows the HCV genotype status of each subject.

TABLE 8
MicroRNA-122 levels in HCV infected subjects.

| | HCV status | |
|---|---|---|
| Subject | (HCV genotype) | miR-122 copies/20 pg |
| HCV 1A 016 | gt 1a | 66.5 |
| HCV 1A 017 | gt 1a | 51.9 |
| HCV 1A 018 | gt 1a | 114.5 |
| HCV 1A 019 | gt 1a | 5.0 |
| HCV 1A 020 | gt 1a | 23.0 |
| HCV 1A 021 | gt 1a | 11.6 |
| HCV 1A 022 | gt 1a | 197.7 |
| HCV 1A 023 | gt 1a | 80.5 |
| HCV 1A 024 | gt 1a | 334.4 |
| HCV 001 | gt 1b | 178.9 |
| HCV 002 | gt 1b | 134.1 |
| HCV 003 | gt 1b | 306.5 |
| HCV 004 | gt 1b | 29.0 |
| HCV 005 | gt 1b | 358.0 |
| HCV 006 | gt 1b | 7.6 |
| HCV 007 | gt 1b | 26.1 |
| HCV 008 | gt 1b | 97.8 |
| HCV 009 | gt 1b | 125.2 |
| HCV 010 | gt 1b | 85.1 |
| A113 | uninfected | 4.7 |
| A114 | uninfected | 16.8 |
| A115 | uninfected | 2.8 |
| A116 | uninfected | 3.3 |
| A225 | uninfected | 5.3 |
| A226 | uninfected | 1.7 |
| A227 | uninfected | 9.9 |
| A228 | uninfected | 7.9 |
| A229 | uninfected | 17.0 |
| A130 | uninfected | 30.8 |
| Average | gt 1A | 98.3 +/− 106 |
| Average | gt 1B | 134.8 +/− 117 |
| Average | uninfected | 10.2 +/− 9.3 |

The data presented in Table 8 shows that although there is wide subject-to-subject variation, the level of miR-122 detected in plasma of HCV-infected subjects was 10-fold or higher on average than in uninfected subjects.

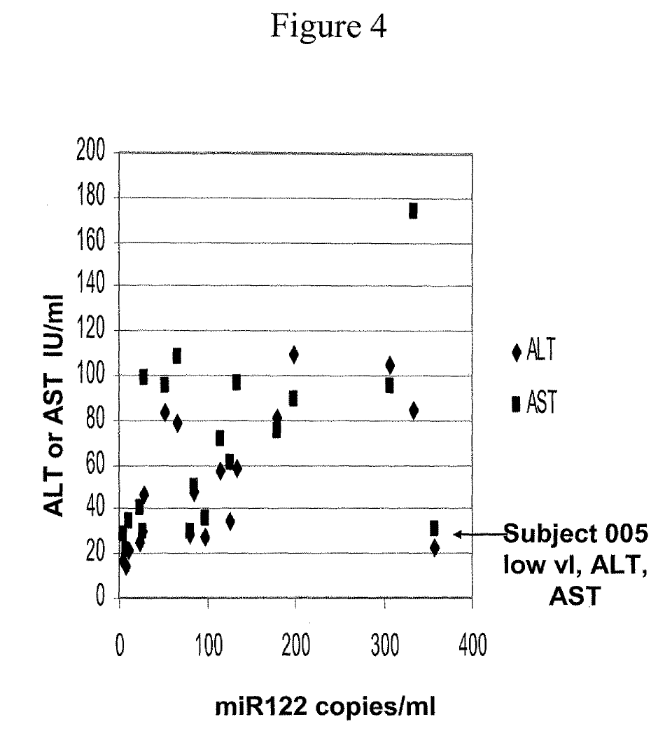

Standard clinical blood chemistry data was obtained for these same subjects. Plasma ALT and AST levels are common protein biomarkers for liver damage. A plot of ALT or AST levels verses miR122 copy number for the samples set forth in Table 8 are displayed in FIG. 4. These data suggest that there is a correlation between plasma miR-122 copy number and ALT or AST levels in this sample set. The single non-correlating data entry for ALT and for AST were both derived from the same subject. This individual had low ALT, low AST, low viral load (<615 IU/ml) but high plasma miR122 copy level. These results show that the level of miR-122 detected in the blood plasma of chronically infected HCV subjects is correlated with chronic HCV infection and resulting liver tissue injury as measured by other accepted biomarkers of injury.

### REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for classifying a vertebrate subject as having tissue injury, said method comprising:
a) obtaining a fluid sample from said subject, wherein the fluid sample is not obtained directly from an organ or tissue that is suspected of being injured;
b) detecting the presence of a miRNA selected from the group consisting of miR-10a, miR-10b, miR-122a, miR-124a, miR-133a, miR-133b, and any combination or sub-combination thereof, in said fluid sample,
wherein the presence of said miRNA is detected when said miRNA has a measured value above a threshold value for said miRNA; and,
c) classifying said vertebrate subject as having tissue injury if said detected miRNA has a measured value above said threshold value.

2. The method of claim 1, wherein said miRNA is miR-122a, said fluid sample is whole blood, blood plasma, or blood serum, and said tissue injury is in the liver.

3. The method of claim 1, wherein said miRNA is miR-10a and/or miR-10b, said fluid sample is urine, and said tissue injury is in the kidney.

4. (canceled)

5. The method of claim 1, wherein said miRNA is miR-133a and/or miR-133b, said fluid sample is whole blood, blood plasma, or blood serum, and said tissue injury is in skeletal muscle or cardiac muscle.

6. The method of claim 1, wherein said miRNA is miR-124a, said fluid sample is whole blood, blood plasma, blood serum or cerebrospinal fluid, and said tissue injury is in the brain.

7. (canceled)

8. The method of claim 1 wherein said subject is suspected of being exposed to an agent that is known to cause tissue injury.

9. The method of claim 8, wherein a control fluid sample is obtained from said subject before said subject is exposed to said agent and said fluid sample is obtained from said subject after said subject is exposed to said agent.

10. The method of claim 8, wherein said agent is a therapeutic agent.

11. The method of claim 8, wherein said agent is an infectious agent.

12. The method of claim 1 wherein said measured value is obtained using a polymerase chain reaction method, a Northern blot hybridization method, or a nucleotide microarray hybridization method.

13. The method of claim 1, wherein said threshold value is obtained by obtaining a second measured value of said miRNA in a control fluid sample obtained from a control vertebrate subject that has not suffered tissue injury.

14. The method of claim 1, wherein said vertebrate subject is selected from the group consisting of dog, cat, pig, rat, mouse, monkey, baboon, ape, human, bison, cow, horse, and chicken.

15. The method of claim 11 wherein said infectious agent is hepatitis C virus.

16. The method of claim 1 additionally comprising:
d) displaying; or outputting to a user interface device, a computer readable medium, or a local or remote computer system, the classification result obtained from step (c).

17. A method of monitoring a vertebrate subject who is exposed to an agent that has a risk of causing tissue damage, said method comprising:
a) obtaining a fluid sample from said subject who is exposed to said agent, wherein the fluid sample is not obtained directly from an organ or tissue that is suspected of being injured by said agent;
b) measuring a level of one or more miRNA in said fluid sample from said subject, wherein said one or more miRNA is selected from the group consisting of miR-10a, miR-10b, miR-122a, miR-124a, miR-133a, miR-133b, and any combination or sub-combination thereof; and,
c) identifying said subject as being at risk of tissue injury or not based on the measured level of the one or more miRNA.

18. The method of claim 17, wherein said miRNA is miR-122a, said fluid sample is whole blood, blood plasma, or blood serum, and the risk of tissue injury is to liver.

19. The method of claim 17, wherein said miRNA is miR-10a and/or miR-10b, said fluid sample is urine, and the risk of tissue injury is to kidney.

20. The method of claim 17, wherein said miRNA is miR-133a and/or miR-133b, said fluid sample is whole blood, blood plasma, or blood serum, and the risk of tissue injury is to skeletal muscle or cardiac muscle.

21. The method of claim 17, wherein said miRNA is miR-124a, said fluid sample is whole blood, blood plasma, blood serum or cerebrospinal fluid, and the risk of tissue injury is to brain.

22-32. (canceled)

33. A method of identifying an agent as having a risk of causing tissue injury to a vertebrate subject, said method comprising:
a) obtaining a fluid sample from a subject exposed to said agent, wherein the fluid sample is not obtained directly from an organ or tissue that is suspected of being injured by said agent;
b) measuring a level of one or more miRNA in said fluid sample from said subject, wherein said one or more miRNA is selected from the group consisting of miR-10a, miR-10b, miR-122a, miR-124a, miR-133a, miR-133b, and any combination or sub-combination thereof; and,
c) identifying said agent as having a risk of causing tissue injury based on said measured level of said one or more miRNA.

34-50. (canceled)


SEQUENCE LISTING (28 sequences)

SEQ ID NO: 1 - RNA, 23 nt, Homo sapiens
uggagugugacaaugguguuugu

SEQ ID NO: 2 - RNA, 22 nt, Homo sapiens
ucacagugaaccggucucuuuu

SEQ ID NO: 3 - RNA, 22 nt, Homo sapiens
ucacagugaaccggucucuuuc

SEQ ID NO: 4 - RNA, 21 nt, Homo sapiens
cuuuuugcggucugggcuugc

SEQ ID NO: 5 - RNA, 22 nt, Homo sapiens
uugguccccuucaaccagcugu

SEQ ID NO: 6 - RNA, 21 nt, Homo sapiens
uugguccccuucaaccagcua

SEQ ID NO: 7 - RNA, 22 nt, Homo sapiens
uauugcuuaagaauacgcguag

SEQ ID NO: 8 - RNA, 23 nt, Homo sapiens
cagugcaauaguauugucaaagc

SEQ ID NO: 9 - RNA, 23 nt, Homo sapiens
uaagugcuuccauguuucagugg

SEQ ID NO: 10 - RNA, 21 nt, Homo sapiens
ggcaagaugcuggcauagcug

SEQ ID NO: 11 - RNA, 22 nt, Homo sapiens
gcacauuacacggucgaccucu

SEQ ID NO: 12 - RNA, 23 nt, Homo sapiens
gcaaagcacacggccugcagaga

SEQ ID NO: 13 - RNA, 23 nt, Homo sapiens
ugucugcccgcaugccugccucu

SEQ ID NO: 14 - RNA, 23 nt, Homo sapiens
uaggcagugucauuagcugauug

SEQ ID NO: 15 - RNA, 23 nt, Homo sapiens
aggcaguguaguuagcugauugc

SEQ ID NO: 16 - RNA, 22 nt, Homo sapiens
uuuguucguucggcucgcguga

SEQ ID NO: 17 - RNA, 21 nt, Homo sapiens
uaaagcuagauaaccgaaagu

SEQ ID NO: 18 - RNA, 22 nt, Homo sapiens
uuaaggcacgcggugaaugcca

SEQ ID NO: 19 - RNA, 23 nt, Homo sapiens
agcugguguugugaaucaggccg

SEQ ID NO: 20 - RNA, 22 nt, Homo sapiens
ucuggcuccgugucuucacucc

SEQ ID NO: 21 - RNA, 20 nt, Homo sapiens
uugcauagucacaaaaguga

SEQ ID NO: 22 - RNA, 22 nt, Homo sapiens
aacauucaaccugucggugagu

SEQ ID NO: 23 - RNA, 22 nt, Homo sapiens
uaaggugcaucuagugcagaua

SEQ ID NO: 24 - RNA, 22 nt, Homo sapiens
uccuucauuccaccggagucug

SEQ ID NO: 25 - RNA, 21 nt, Homo sapiens
uaaucucagcuggcaacugug

SEQ ID NO: 26 - RNA, 24 nt, Homo sapiens
uacugcaucaggaacugauuggau

SEQ ID NO: 27 - RNA, 23 nt, Homo sapiens
uccagcaucagugauuuuguuga

SEQ ID NO: 28 - RNA, 22 nt, Homo sapiens
agaucagaaggugauuguggcu